(19) United States
(12) Patent Application Publication
(10) Pub. No.: US 2011/0020941 A1
(43) Pub. Date: Jan. 27, 2011

(54) GLYCOSYLATION MARKERS FOR PANCREATITIS, SEPSIS AND PANCREATIC CANCER

(75) Inventors: Pauline Rudd, Dublin (IE); Louise Royle, Oxfordshire (GB); Gordon Lauc, Zagreb (HR); Olga Gornick, Zagreb (HR)

Correspondence Address: FOLEY AND LARDNER LLP;SUITE 500, 3000 K STREET NW, WASHINGTON, DC, 20007, US

(73) Assignee: National Institute for Bioprocessing Research and Training Limited

(21) Appl. No.: 12/733,937

(22) PCT Filed: Oct. 6, 2008
(86) PCT No.: PCT/GB2008/050912

§ 371 Date: Oct. 1, 2010

Related U.S. Application Data
(60) Provisional application No. 60/977,842, filed on Oct. 5, 2007.

(30) Foreign Application Priority Data
Oct. 5, 2007 (IE) 2007/0718

(51) Int. Cl. G01N 33/48

(52) U.S. Cl. 436/63

(57) ABSTRACT

The present invention provides novel biomarkers for use in the diagnosis and prognosis of cancer and chronic inflammation and further of diseases which are mediated by chronic inflammation. The biomarkers are glycoproteins, the levels of which have been correlated by the inventors to correspond to particular disease conditions. The invention further extends to methods for monitoring the response to therapy of a treatment of a cancerous or chronic inflammatory condition.

### FIELD OF THE INVENTION

The present invention relates to methods of diagnosing and monitoring diseases based on analysis of glycosylation. In particular, the invention relates to methods of diagnosing and monitoring sepsis, pancreatitis and pancreatic cancer.

### BACKGROUND TO THE INVENTION

The inflammatory response comprises a sequence of cellular and molecular events which occur in response to stimuli such as infection or tissue damage. However, differences in patterns of immune mediators, such as cytokines, prostaglandins and leukotrienes during acute phase responses occur in different pathophysiological conditions depending upon the nature and the site of inflammation. Changes in glycan structures during the onset and progression of disease are exceedingly complex and poorly understood processes that involve changes in the expression of glycosyltransferases, their intracellular localization and stability, the availability and transport of activated sugar nucleotides as well as intracellular trafficking of glycoprotein acceptors. Changes in glycosylation during inflammation have been demonstrated in animal models and on some individual human acute-phase proteins. Altered oligosaccharide branching and increased sialylation of α-1 acid glycoprotein as well as altered galactosylation of the immunoglobulin IgG in different inflammatory diseases are prominent examples.

Sepsis is a clinical condition which results from a systemic response to infection. Since the majority of pathophysiological events during sepsis result from overreaction or uncontrolled inflammatory response, any contribution to the understanding of these processes would have value in developing therapeutic treatments.

Pancreatitis is an inflammatory disease of the pancreatic tissue which is caused by activation of enzymes within the pancreas. Acute pancreatitis involves a systemic inflammatory response, however no bacterial infection is observed during the initial stages of disease development. Early diagnosis of pancreatitis is important, as prompt treatment can reduce the risk of later complications. However, at present there is a distinct lack of reliable prognostic markers which can be used to predict the onset and progression of this potentially life-threatening disease.

Despite the widely accepted fact that glycosylation is essential in the process of inflammation, studies of glycosylation changes in sepsis are scarce, while glycosylation changes in pancreatitis have not been addressed to date.

There is therefore a substantial need for the identification of markers which are specific to either sepsis or pancreatitis, wherein the markers allow for prognosis and diagnosis of these conditions. Such markers may have further utility in monitoring disease progress, or further, in monitoring a response to a therapy used to treat each of these conditions.

Following extensive experimentation, the inventors have identified a number of defined changes in glycosylation which have utility in methods for the diagnosis or prognosis of sepsis, or further in methods for assessing the response to therapy in a subject who has been administered a therapeutic treatment for sepsis. The identification and characterisation of these changes in glycosylation provides disease specific markers which have particular utility in the provision of improved methods for diagnosing these conditions and for monitoring their development, particularly in response to therapy.

### SUMMARY OF THE INVENTION

According to a first aspect of the present invention, there is provided a method for the diagnosis of sepsis, the method comprising the steps of:
providing a test sample from a subject;
determining the level of at least one marker selected from the group consisting of outer arm to core fucosylation on N-linked glycans, trisialylated N-linked glycans, tetrasialylated N-linked glycans, biantennary glycans, mannose structures on N-linked glycans, degree of branching of N-linked glycans, ratio of trisialylated N-linked glycan A3G3S3 to fucosylated N-linked glycan A3FG3S3 (sialyl Lewis x), ratio of alpha 1,3 fucosylated forms of N-linked glycans to core fucosylated or non-fucosylated forms of N-linked glycans, trisialylated triantennary N-linked glycan A3G3S3, sialyl Lewis x N-linked glycan structure A3FG3S3, A3FG1 derived from digestion of sialyl Lewis x on N-linked glycans, oligomannose structures on N-linked glycans, isoforms of triantennary glycans with a branched 6-antenna and core fucosylation of N-linked glycans in the test sample; and
providing a diagnosis based on said determined level of the at least one marker;

wherein the diagnosis confirms the presence or absence of sepsis in the subject.

In certain embodiments, the sepsis is caused by a gram positive bacteria. In certain embodiments, the sepsis is gram negative sepsis.

The inventors have further recognised the utility of the identified biomarkers in diagnosing sepsis.

Accordingly, in a further aspect of the invention, there is provided the use of at least one glycosylation marker selected from the group consisting of outer arm to core fucosylation on N-linked glycans, trisialylated N-linked glycans, tetrasialylated N-linked glycans, biantennary glycans, mannose structures on N-linked glycans, degree of branching of N-linked glycans, ratio of trisialylated N-linked glycan A3G3S3 to fucosylated N-linked glycan A3FG3S3 (sialyl Lewis x), ratio of alpha 1,3 fucosylated forms of N-linked glycans to core fucosylated or non-fucosylated forms of N-linked glycans, trisialylated triantennary N-linked glycan A3G3S3, sialyl Lewis x N-linked glycan structure A3FG3S3, A3FG1 derived from digestion of sialyl Lewis x on N-linked glycans, oligomannose structures on N-linked glycans, isoforms of triantennary glycans with a branched 6-antenna and core fucosylation of N-linked glycans in diagnosing a condition defined as sepsis in a subject.

In certain embodiments, the sepsis is caused by a gram positive bacteria. In certain embodiments, the sepsis is gram negative sepsis.

The inventors have further identified that, in addition to the diagnosis of sepsis, the method and markers of the first aspect of the invention have further utility in methods for the prognosis of sepsis in a subject.

Accordingly, a further aspect of the present invention provides a method for the prognosis of sepsis in a subject, the method comprising the steps of:
providing a test sample from a subject;
determining the level of at least one marker selected from the group consisting of outer arm to core fucosylation on N-linked glycans, trisialylated N-linked glycans, tetrasialylated N-linked glycans, biantennary glycans, mannose structures on N-linked glycans, degree of branching of N-linked glycans, ratio of trisialylated N-linked glycan A3G3S3 to fucosylated N-linked glycan A3FG3S3 (sialyl Lewis x), ratio of alpha 1,3 fucosylated forms of N-linked glycans to core fucosylated or non-fucosylated forms of N-linked glycans, trisialylated triantennary N-linked glycan A3G3S3, sialyl Lewis x N-linked glycan structure A3FG3S3, A3FG1 derived from digestion of sialyl Lewis x on N-linked glycans, oligomannose structures on N-linked glycans, isoforms of triantennary glycans with a branched 6-antenna and core fucosylation of N-linked glycans in the test sample; and
providing a prognosis based on said determined level of the at least one marker;

wherein the prognosis confirms the possibility of the subject developing sepsis.

In certain embodiments, the sepsis is caused by a gram positive bacteria. In certain embodiments, the sepsis is gram negative sepsis.

The inventors have further recognised the utility of the identified biomarkers in the prognosis of sepsis.

Accordingly, in a further aspect of the invention, there is provided the use of at least one glycosylation marker selected from the group consisting of outer arm to core fucosylation on N-linked glycans, trisialylated N-linked glycans, tetrasialylated N-linked glycans, biantennary glycans, mannose structures on N-linked glycans, degree of branching of N-linked glycans, ratio of trisialylated N-linked glycan A3G3S3 to fucosylated N-linked glycan A3FG3S3 (sialyl Lewis x), ratio of alpha 1,3 fucosylated forms of N-linked glycans to core fucosylated or non-fucosylated forms of N-linked glycans, trisialylated triantennary N-linked glycan A3G3S3, sialyl Lewis x N-linked glycan structure A3FG3S3, A3FG1 derived from digestion of sialyl Lewis x on N-linked glycans, oligomannose structures on N-linked glycans, isoforms of triantennary glycans with a branched 6-antenna and core fucosylation of N-linked glycans in the prognosis of sepsis.

In certain embodiments, the sepsis is caused by a gram positive bacteria. In certain embodiments, the sepsis is gram negative sepsis.

Furthermore, the markers and methods of the present invention have utility in methods for monitoring the response by a subject to a therapeutic treatment, which is or has been administered to a subject for the purpose of the treatment of sepsis or for the amelioration of at least one symptom associated therewith.

Accordingly, a yet further aspect of the present invention provides a method for monitoring the response to a treatment for sepsis comprising:
providing a first test sample from a subject obtained at a first time point;
determining the level of at least one marker in said sample, the marker being selected from the group consisting of at least one of: outer arm to core fucosylation on N-linked glycans, trisialylated N-linked glycans, tetrasialylated N-linked glycans, biantennary glycans, mannose structures on N-linked glycans, degree of branching of N-linked glycans, ratio of trisialylated N-linked glycan A3G3S3 to fucosylated N-linked glycan A3FG3S3 (sialyl Lewis x), ratio of alpha 1,3 fucosylated forms of N-linked glycans to core fucosylated or non-fucosylated forms of N-linked glycans, trisialylated triantennary N-linked glycan A3G3S3, sialyl Lewis x N-linked glycan structure A3FG3S3, A3FG1 derived from digestion of sialyl Lewis x on N-linked glycans, oligomannose structures on N-linked glycans, isoforms of triantennary glycans with a branched 6-antenna or core fucosylation of N-linked glycans;
providing at least one further test sample from said subject at a time point after said first sample was obtained;
determining the level of at least one marker selected from the group consisting of outer arm to core fucosylation on N-linked glycans, trisialylated N-linked glycans, tetrasialylated N-linked glycans, biantennary glycans, mannose structures on N-linked glycans, degree of branching of N-linked glycans, ratio of trisialylated N-linked glycan A3G3S3 to fucosylated N-linked glycan A3FG3S3 (sialyl Lewis x), ratio of alpha 1,3 fucosylated forms of N-linked glycans to core fucosylated or non-fucosylated forms of N-linked glycans, trisialylated triantennary N-linked glycan A3G3S3, sialyl Lewis x N-linked glycan structure A3FG3S3, A3FG1 derived from digestion of sialyl Lewis x on N-linked glycans, oligomannose structures on N-linked glycans, isoforms of triantennary glycans with a branched 6-antenna or core fucosylation of N-linked glycans in the at least one further test sample; and
providing an assessment of response to therapy based on a comparison of the determined level of the at least one marker which is present in said first sample when compared to the level of the same at least one marker in the said at least one further sample.

In certain embodiments, the sepsis is caused by a gram positive bacteria. In certain embodiments, the sepsis is gram negative sepsis.

The inventors have further recognised the utility of the identified biomarkers in determining the response to therapy by a subject to a therapeutic treatment regime which is provided to the subject to treat sepsis.

Accordingly, in a further aspect of the invention, there is provided the use of at least one glycosylation marker selected from the group consisting of outer arm to core fucosylation on N-linked glycans, trisialylated N-linked glycans, tetrasialylated N-linked glycans, biantennary glycans, mannose structures on N-linked glycans, degree of branching of N-linked glycans, ratio of trisialylated N-linked glycan A3G3S3 to fucosylated N-linked glycan A3FG3S3 (sialyl Lewis x), ratio of alpha 1,3 fucosylated forms of N-linked glycans to core fucosylated or non-fucosylated forms of N-linked glycans, trisialylated triantennary N-linked glycan A3G3S3, sialyl Lewis x N-linked glycan structure A3FG3S3, A3FG1 derived from digestion of sialyl Lewis x on N-linked glycans, oligomannose structures on N-linked glycans, isoforms of triantennary glycans with a branched 6-antenna and core fucosylation of N-linked glycans in a method for determining the response of a subject to a treatment administered to said subject to treat sepsis.

In certain embodiments, the sepsis is caused by a gram positive bacteria. In certain embodiments, the sepsis is gram negative sepsis.

In certain embodiments where a diagnosis, prognosis, or evaluation of response to therapy is provided by at least one of the methods of the invention in relation to sepsis, the diagnosis or prognosis may determine factors such as whether the subject has sepsis, whether the subject has gram positive sepsis, whether the subject does not have sepsis or whether the subject has gram negative sepsis.

The inventors have further identified glycosylation based biomarkers which have utility in methods for the diagnosis or prognosis of pancreatitis, or further in methods for assessing the response to therapy in a subject who has been administered a therapeutic treatment for pancreatitis.

According to a further aspect of the present invention, there is provided a method for the diagnosis of pancreatitis, the method comprising the steps of:
providing a test sample from a subject;
determining the level of at least one marker selected from the group consisting of outer arm to core fucosylation on N-linked glycans, trisialylated N-linked glycans, tetrasialylated N-linked glycans, biantennary glycans, mannose structures on N-linked glycans, degree of branching of N-linked glycans, ratio of trisialylated N-linked glycan A3G3S3 to fucosylated N-linked glycan A3FG3S3 (sialyl Lewis x), ratio of alpha 1,3 fucosylated forms of N-linked glycans to core fucosylated or non-fucosylated forms of N-linked glycans, trisialylated triantennary N-linked glycan A3G3S3, sialyl Lewis x N-linked glycan structure A3FG3S3, A3FG1 derived from digestion of sialyl Lewis x on N-linked glycans, oligomannose structures on N-linked glycans, isoforms of triantennary glycans with a branched 6-antenna and core fucosylation of N-linked glycans in the sample; and
providing a diagnosis based on said determined level of the at least one marker;

wherein the diagnosis confirms the presence or absence of pancreatitis in the subject.

The inventors have further recognised the utility of the identified biomarkers in diagnosing pancreatitis.

Accordingly, in a further aspect of the invention, there is provided the use of at least one glycosylation marker selected from the group consisting of: outer arm to core fucosylation on N-linked glycans, trisialylated N-linked glycans, tetrasialylated N-linked glycans, biantennary glycans, mannose structures on N-linked glycans, degree of branching of N-linked glycans, ratio of trisialylated N-linked glycan A3G3S3 to fucosylated N-linked glycan A3FG3S3 (sialyl Lewis x), ratio of alpha 1,3 fucosylated forms of N-linked glycans to core fucosylated or non-fucosylated forms of N-linked glycans, trisialylated triantennary N-linked glycan A3G3S3, sialyl Lewis x N-linked glycan structure A3FG3S3, A3FG1 derived from digestion of sialyl Lewis x on N-linked glycans, oligomannose structures on N-linked glycans, isoforms of triantennary glycans with a branched 6-antenna and core fucosylation of N-linked glycans in diagnosing pancreatitis.

The inventors have further identified that, in addition to the diagnosis of pancreatitis, the method and markers of the invention have further utility in methods for the prognosis of pancreatitis in a subject.

Accordingly, a further aspect of the present invention provides a method for the prognosis of pancreatitis in a subject, the method comprising the steps of:
providing a test sample from a subject;
determining the level of at least one marker selected from the group consisting of outer arm to core fucosylation on N-linked glycans, trisialylated N-linked glycans, tetrasialylated N-linked glycans, biantennary glycans, mannose structures on N-linked glycans, degree of branching of N-linked glycans, ratio of trisialylated N-linked glycan A3G3S3 to fucosylated N-linked glycan A3FG3S3 (sialyl Lewis x), ratio of alpha 1,3 fucosylated forms of N-linked glycans to core fucosylated or non-fucosylated forms of N-linked glycans, trisialylated triantennary N-linked glycan A3G3S3, sialyl Lewis x N-linked glycan structure A3FG3S3, A3FG1 derived from digestion of sialyl Lewis x on N-linked glycans, oligomannose structures on N-linked glycans, isoforms of triantennary glycans with a branched 6-antenna and core fucosylation of N-linked glycans in the sample; and
providing a prognosis based on said determined level of the at least one marker;

wherein the prognosis confirms the possibility of the subject developing pancreatitis.

The inventors have further recognised the utility of the identified biomarkers in the prognosis of pancreatitis.

Accordingly, in a further aspect of the invention, there is provided the use of at least one glycosylation marker selected from the group consisting of outer arm to core fucosylation on N-linked glycans, trisialylated N-linked glycans, tetrasialylated N-linked glycans, biantennary glycans, mannose structures on N-linked glycans, degree of branching of N-linked glycans, ratio of trisialylated N-linked glycan A3G3S3 to fucosylated N-linked glycan A3FG3S3 (sialyl Lewis x), ratio of alpha 1,3 fucosylated forms of N-linked glycans to core fucosylated or non-fucosylated forms of N-linked glycans, trisialylated triantennary N-linked glycan A3G3S3, sialyl Lewis x N-linked glycan structure A3FG3S3, A3FG1 derived from digestion of sialyl Lewis x on N-linked glycans, oligomannose structures on N-linked glycans, isoforms of triantennary glycans with a branched 6-antenna and core fucosylation of N-linked glycans in the prognosis of pancreatitis.

Furthermore, the markers and methods of the present invention have utility in methods for monitoring the response by a subject to a therapeutic treatment for pancreatitis.

Accordingly, a yet further aspect of the present invention provides a method for monitoring the response to a treatment for pancreatitis comprising:
providing a first test sample from a subject obtained at a first time point;
determining the level of at least one marker selected from the group consisting of outer arm to core fucosylation on N-linked glycans, trisialylated N-linked glycans, tetrasialylated N-linked glycans, biantennary glycans, mannose structures on N-linked glycans, degree of branching of N-linked glycans, ratio of trisialylated N-linked glycan A3G3S3 to fucosylated N-linked glycan A3FG3S3 (sialyl Lewis x), ratio of alpha 1,3 fucosylated forms of N-linked glycans to core fucosylated or non-fucosylated forms of N-linked glycans, trisialylated triantennary N-linked glycan A3G3S3, sialyl Lewis x N-linked glycan structure A3FG3S3, A3FG1 derived from digestion of sialyl Lewis x on N-linked glycans, oligomannose structures on N-linked glycans, isoforms of triantennary glycans with a branched 6-antenna and core fucosylation of N-linked glycans;
providing at least one further test sample from said subject at a time point after said first sample was obtained;
determining the level of at least one marker selected from the group consisting of outer arm to core fucosylation on N-linked glycans, trisialylated N-linked glycans, tetrasialylated N-linked glycans, biantennary glycans, mannose structures on N-linked glycans, degree of branching of N-linked glycans, ratio of trisialylated N-linked glycan A3G3S3 to fucosylated N-linked glycan A3FG3S3 (sialyl Lewis x), ratio of alpha 1,3 fucosylated forms of N-linked glycans to core fucosylated or non-fucosylated forms of N-linked glycans, trisialylated triantennary N-linked glycan A3G3S3, sialyl Lewis x N-linked glycan structure A3FG3S3, A3FG1 derived from digestion of sialyl Lewis x on N-linked glycans, oligomannose structures on N-linked glycans, isoforms of triantennary glycans with a branched 6-antenna and core fucosylation of N-linked glycans in the at least one further test sample; and
providing an assessment of response to therapy based on a comparison of the determined level of the at least one marker which is present in said first sample when compared to the level of the same at least one marker in the said at least one further sample.

The inventors have further recognised the utility of the identified biomarkers in the determining the response to therapy by a subject to a therapeutic treatment regime which is provided to the subject to treat pancreatitis.

Accordingly, in a further aspect of the invention, there is provided the use of at least one glycosylation marker selected from the group consisting of outer arm to core fucosylation on N-linked glycans, trisialylated N-linked glycans, tetrasialylated N-linked glycans, biantennary glycans, mannose structures on N-linked glycans, degree of branching of N-linked glycans, ratio of trisialylated N-linked glycan A3G3S3 to fucosylated N-linked glycan A3FG3S3 (sialyl Lewis x), ratio of alpha 1,3 fucosylated forms of N-linked glycans to core fucosylated or non-fucosylated forms of N-linked glycans, trisialylated triantennary N-linked glycan A3G3S3, sialyl Lewis x N-linked glycan structure A3FG3S3, A3FG1 derived from digestion of sialyl Lewis x on N-linked glycans, oligomannose structures on N-linked glycans, isoforms of triantennary glycans with a branched 6-antenna and core fucosylation of N-linked glycans for determining the response of a subject to a treatment administered to said subject to treat pancreatitis.

In certain embodiments where a diagnosis, prognosis, or evaluation of response to therapy is provided by at least one of the methods of the invention in relation to pancreatitis, the diagnosis or prognosis may determine factors such as whether the subject has pancreatitis, or acute pancreatitis, whether the subject does not have pancreatitis and further, may have utility in determining or assisting in the determination of a characterisation of the stage of disease progression and/or severity.

The inventors have further identified glycosylation based biomarkers which have utility in methods for the diagnosis or prognosis of pancreatic cancer, or further in methods for assessing the response to therapy in a subject who has been administered a therapeutic treatment for pancreatic cancer.

According to a further aspect of the present invention, there is provided a method for the diagnosis of pancreatic cancer, the method comprising the steps of:
providing a test sample from a subject;
determining the level of at least one marker selected from the group consisting of: outer arm to core fucosylation on N-linked glycans, trisialylated N-linked glycans, tetrasialylated N-linked glycans, biantennary glycans, mannose structures on N-linked glycans, degree of branching of N-linked glycans, ratio of trisialylated N-linked glycan A3G3S3 to fucosylated N-linked glycan A3FG3S3 (sialyl Lewis x), ratio of alpha 1,3 fucosylated forms of N-linked glycans to core fucosylated or non-fucosylated forms of N-linked glycans, trisialylated triantennary N-linked glycan A3G3S3, sialyl Lewis x N-linked glycan structure A3FG3S3, A3FG1 derived from digestion of sialyl Lewis x on N-linked glycans, oligomannose structures on N-linked glycans, isoforms of triantennary glycans with a branched 6-antenna and core fucosylation of N-linked glycans present in said sample; and
providing a diagnosis based on said determined level of the at least one marker;

wherein the diagnosis confirms the presence or absence of pancreatic cancer in the subject.

The inventors have further recognised the utility of the identified biomarkers in methods for diagnosing pancreatic cancer.

Accordingly, in a further aspect of the invention, there is provided the use of at least one glycosylation marker selected from the group consisting of outer arm to core fucosylation on N-linked glycans, trisialylated N-linked glycans, tetrasialylated N-linked glycans, biantennary glycans, mannose structures on N-linked glycans, degree of branching of N-linked glycans, ratio of trisialylated N-linked glycan A3G3S3 to fucosylated N-linked glycan A3FG3S3 (sialyl Lewis x), ratio of alpha 1,3 fucosylated forms of N-linked glycans to core fucosylated or non-fucosylated forms of N-linked glycans, trisialylated triantennary N-linked glycan A3G3S3, sialyl Lewis x N-linked glycan structure A3FG3S3, A3FG1 derived from digestion of sialyl Lewis x on N-linked glycans, oligomannose structures on N-linked glycans, isoforms of triantennary glycans with a branched 6-antenna or core fucosylation of N-linked glycans in diagnosing pancreatic cancer.

The inventors have further identified that, in addition to the diagnosis of pancreatic cancer, the method and markers of the invention have further utility in methods for the prognosis of pancreatic cancer in a subject.

Accordingly, a further aspect of the present invention provides a method for the prognosis of pancreatic cancer in a subject, the method comprising the steps of:
providing a test sample from a subject;
determining the level of at least one marker selected from the group consisting of outer arm to core fucosylation on N-linked glycans, trisialylated N-linked glycans, tetrasialylated N-linked glycans, biantennary glycans, mannose structures on N-linked glycans, degree of branching of N-linked glycans, ratio of trisialylated N-linked glycan A3G3S3 to fucosylated N-linked glycan A3FG3S3 (sialyl Lewis x), ratio of alpha 1,3 fucosylated forms of N-linked glycans to core fucosylated or non-fucosylated forms of N-linked glycans, trisialylated triantennary N-linked glycan A3G3S3, sialyl Lewis x N-linked glycan structure A3FG3S3, A3FG1 derived from digestion of sialyl Lewis x on N-linked glycans, oligomannose structures on N-linked glycans, isoforms of triantennary glycans with a branched 6-antenna or core fucosylation of N-linked glycans; and
providing a prognosis based on said determined level of the at least one marker;

wherein the prognosis confirms the possibility of the subject developing pancreatic cancer.

The inventors have further recognised the utility of the identified biomarkers in the prognosis of pancreatic cancer.

Accordingly, in a further aspect of the invention, there is provided the use of at least one glycosylation marker selected from the group consisting of outer arm to core fucosylation on N-linked glycans, trisialylated N-linked glycans, tetrasialylated N-linked glycans, biantennary glycans, mannose structures on N-linked glycans, degree of branching of N-linked glycans, ratio of trisialylated N-linked glycan A3G3S3 to fucosylated N-linked glycan A3FG3S3 (sialyl Lewis x), ratio of alpha 1,3 fucosylated forms of N-linked glycans to core fucosylated or non-fucosylated forms of N-linked glycans, trisialylated triantennary N-linked glycan A3G3S3, sialyl Lewis x N-linked glycan structure A3FG3S3, A3FG1 derived from digestion of sialyl Lewis x on N-linked glycans, oligomannose structures on N-linked glycans, isoforms of triantennary glycans with a branched 6-antenna and core fucosylation of N-linked glycans in the prognosis of pancreatic cancer.

Furthermore, the markers and methods of the present invention have utility in methods for monitoring the response by a subject to a therapeutic treatment for pancreatic cancer.

Accordingly, a yet further aspect of the present invention provides a method for monitoring the response to therapy of a treatment for pancreatic cancer comprising:
providing a first test sample from a subject obtained at a first time point;
determining the level of at least one marker selected from the group consisting of outer arm to core fucosylation on N-linked glycans, trisialylated N-linked glycans, tetrasialylated N-linked glycans, biantennary glycans, mannose structures on N-linked glycans, degree of branching of N-linked glycans, ratio of trisialylated N-linked glycan A3G3S3 to fucosylated N-linked glycan A3FG3S3 (sialyl Lewis x), ratio of alpha 1,3 fucosylated forms of N-linked glycans to core fucosylated or non-fucosylated forms of N-linked glycans, trisialylated triantennary N-linked glycan A3G3S3, sialyl Lewis x N-linked glycan structure A3FG3S3, A3FG1 derived from digestion of sialyl Lewis x on N-linked glycans, oligomannose structures on N-linked glycans, isoforms of triantennary glycans with a branched 6-antenna or core fucosylation of N-linked glycans;
providing at least one further test sample from said subject at a time point after said first sample was obtained;
determining the level of at least one marker selected from the group consisting of outer arm to core fucosylation on N-linked glycans, trisialylated N-linked glycans, tetrasialylated N-linked glycans, biantennary glycans, mannose structures on N-linked glycans, degree of branching of N-linked glycans, ratio of trisialylated N-linked glycan A3G3S3 to fucosylated N-linked glycan A3FG3S3 (sialyl Lewis x), ratio of alpha 1,3 fucosylated forms of N-linked glycans to core fucosylated or non-fucosylated forms of N-linked glycans, trisialylated triantennary N-linked glycan A3G3S3, sialyl Lewis x N-linked glycan structure A3FG3S3, A3FG1 derived from digestion of sialyl Lewis x on N-linked glycans, oligomannose structures on N-linked glycans, isoforms of triantennary glycans with a branched 6-antenna or core fucosylation of N-linked glycans in the at least one further test sample; and
providing an assessment of response to therapy based on a comparison of the determined level of the at least one marker which is present in said first sample when compared to the level of the same at least one marker in the said at least one further sample.

The inventors have further recognised the utility of the identified biomarkers in determining the response to therapy by a subject to a therapeutic treatment regime which is provided to the subject to treat pancreatic cancer.

Accordingly, in a further aspect of the invention, there is provided the use of at least one glycosylation marker selected from the group consisting of outer arm to core fucosylation on N-linked glycans, trisialylated N-linked glycans, tetrasialylated N-linked glycans, biantennary glycans, mannose structures on N-linked glycans, degree of branching of N-linked glycans, ratio of trisialylated N-linked glycan A3G3S3 to fucosylated N-linked glycan A3FG3S3 (sialyl Lewis x), ratio of alpha 1,3 fucosylated forms of N-linked glycans to core fucosylated or non-fucosylated forms of N-linked glycans, trisialylated triantennary N-linked glycan A3G3S3, sialyl Lewis x N-linked glycan structure A3FG3S3, A3FG1 derived from digestion of sialyl Lewis x on N-linked glycans, oligomannose structures on N-linked glycans, isoforms of triantennary glycans with a branched 6-antenna and core fucosylation of N-linked glycans in determining the response of a subject to a treatment administered to said subject to treat pancreatic cancer.

In certain embodiments where a diagnosis, prognosis, or evaluation of response to therapy is provided by at least one of the methods of the invention in relation to pancreatic cancer, the diagnosis or prognosis may determine factors such as whether the subject has pancreatic cancer, or acute pancreatic cancer, whether the subject does not have pancreatic cancer and further, may have utility in determining or assisting in the determination of a characterisation of the stage of disease progression and/or severity.

In certain embodiments, the subject is a mammal, typically a human.

In certain embodiments, the test sample is obtained by essentially any suitable technique known in the art, and can include, but is not limited to, a sample of body fluid, body tissue, or other sample comprising glycoproteins. Further examples include, but are not limited to, whole serum, blood plasma, blood, urine, sputum, seminal fluid, seminal plasma, pleural fluid, ascites, nipple aspirate, faeces or saliva.

In embodiments of the invention relating to the diagnosis or prognosis of pancreatitis or pancreatic cancer, typically, pancreatic tissue is used. Alternatively, in certain embodiments, a sample can be provided which has been derived from tumour cells.

In certain embodiments, the level of the one or more markers in the test sample is compared with a level of the one or more markers in a control sample to determine the diagnosis, prognosis and/or response.

For example, in one embodiment, an increase in the level of one or more of the ratio of outer arm to core fucosylation on N-linked glycans, tetrasialylated N-linked glycans, biantennary glycans, the ratio of alpha 1,3 fucosylated forms of N-linked glycans to core fucosylated or non-fucosylated forms of N-linked glycans, isoforms of triantennary glycans with a branched 6-antenna, sialyl Lewis x N-linked structure glycan A3FG3S3 and A3FG1 derived from digestion of sialyl Lewis x on N-linked glycans; changes in one or more of the levels mannose structures on N-linked glycans and the degree of branching of N-linked glycans; and/or a decrease in the level of one or more of the trisialylated triantennary N-linked glycan A3G3S3 and the ratio of trisialylated N-linked glycan A3G3S3 to fucosylated N-linked glycan A3FG3S3 (sialyl Lewis x) indicates the presence of pancreatitis and/or sepsis.

The determination of the level in the test sample of the one or more markers may further enable pancreatitis to be distinguished from sepsis. For example, in one embodiment, a continuous increase in the level of the sialyl Lewis x N-linked glycan structure A3FG3S3 and/or a continuous decrease in the level of oligomannose structures is indicative of pancreatitis. The terms “continuous increase”/“continuous decrease” are understood herein to mean that levels continue to increase/decrease over a period of time. A typical period of time over which levels may be assessed is two days, more preferably four days and even more preferably eight days or more.

The determination of the level in the test sample of the one or more markers may further enable pancreatitis and/or sepsis to be distinguished from cancer, for example pancreatic cancer. In one embodiment, an increase in the ratio of alpha 1,3 fucosylated forms of N-linked glycans to core fucosylated or non-fucosylated forms of N-linked glycans is indicative of either pancreatitis or sepsis but not cancer. In one embodiment, an increase in core fucosylation of N-linked glycans is indicative of pancreatic cancer but not pancreatitis or sepsis.

The markers can be detected, for example, from the whole sample, from a pool of glycoproteins from the sample or on one or more proteins purified from the sample.

Thus, in one embodiment, a pool of N-linked glycans is released from total glycoproteins in the test sample (e.g., from serum without purifying the glycoproteins by digestion with a glycosidase) and the level of the one or more markers in the pool of glycans is determined. The glycan markers are optionally detected on particular proteins, for example, on one or more acute phase proteins (e.g., serum amyloid A, haptoglobin, α1-acid glycoprotein, α1-antitrypsin, α1-antichymotrypsin, fibrinogen, transferrin, complement C3, α2-macroglobulin, prothrombin, factor VIII, von Willebrand factor or plasminogen) or other serum protein(s) of interest.

The markers on particular proteins can be detected with or without purification of the proteins from the sample. Thus, in one embodiment, one or more proteins (e.g., one or more acute phase proteins) are isolated from the test sample prior to determining the level of the one or more markers on the proteins. Affinity purification of acute phase proteins to isolate them prior to high-throughput analysis of glycan markers by HPLC is described in the examples herein. The sample can be treated as necessary prior to detection of the markers, for example, cells and/or tissues are optionally lysed for detection of intracellular glycoprotein markers.

The level in the test sample of the one or more markers can be determined by essentially any convenient technique or combination of techniques. For example, the markers can be detected by performing chromatography (e.g., normal phase or weak anion exchange HPLC), mass spectrometry, gel electrophoresis (e.g., one or two dimensional gel electrophoresis) and/or an immunoassay (e.g., immuno-PCR, ELISA, lectin ELISA, Western blot, or lectin immunoassay) on the sample or a derivative or component thereof (e.g., serum, a serum fraction, a cell or tissue lysate, a glycan pool, an isolated protein, etc.). See, e.g., the examples hereinbelow, as well as U.S. patent application publications 20060269974 by Dwek et al. entitled “Glycosylation markers for cancer diagnosing and monitoring”, 20060270048 by Dwek et al. entitled “Automated strategy for identifying physiological glycosylation marker(s),” and 20060269979 by Dwek et al. entitled “High throughput glycan analysis for diagnosing and monitoring rheumatoid arthritis and other autoimmune diseases.”

As set forth in the foregoing embodiments of the invention, various aspects of the invention extend to methods which have utility in monitoring the response of a subject to treatment. Thus, in one class of embodiments wherein the subject has previously been diagnosed with a condition selected from the group consisting of pancreatitis, sepsis and pancreatic cancer, the methods include treating the subject for the condition, providing a first test sample from the subject prior to initiation of the treatment and a second test sample from the subject after initiation of the treatment and comparing the level of the one or more markers in the first test sample with that in the second test sample to monitor the subject's response to the treatment.

Optionally, the level in the test sample of two or more (e.g., three, four, five or six or more) of the markers described herein is determined. Similarly, the markers described herein can be used in combination with other markers for the condition, e.g., glycosylation, genetic and/or protein markers. Thus, for example, the methods can include determining a level in the test sample, or in another clinical sample from the subject, of one or more additional markers and determining the diagnosis, prognosis and/or response from the level of the one or more markers and the level of the one or more additional markers.

The methods optionally include diagnosing, prognosing and/or monitoring response to treatment of pancreatitis, sepsis or pancreatic cancer in the subject based on the level of the one or more markers.

Compositions are another feature of the invention, e.g., compositions useful in practicing, or formed while practicing, the methods of the invention. For example, a composition of the invention optionally includes an antibody against one of the markers of the invention, optionally in combination with other reagents for determining the level of the marker in a sample.

Thus, one exemplary general class of embodiments provide a composition that includes a first antibody against a first glycoform of a first protein, which glycoform comprises one or more of trisialylated N-linked glycan, tetrasialylated N-linked glycan, trisialylated N-linked glycan A3G3S3, fucosylated N-linked glycan A3FG3S3 (sialyl Lewis x), A3FG1 derived from digestion of sialyl Lewis x on N-linked glycans, isoforms of triantennary glycans with a branched 6-antenna, alpha 1,3 fucosylated form of N-linked glycan and core fucosylated and non-fucosylated form of N-linked glycan.

The composition optionally includes the first glycoform of the first protein (e.g., an acute phase protein or other serum protein or protein of interest), a sample from a subject, a lectin, a secondary antibody against the first antibody, a nucleic acid tag associated with the first antibody (covalently or noncovalently, and optionally distinguishable from any other tags on other antibodies in the composition for multiplex assays), a second antibody against a second glycoform of the first protein, and/or a third antibody against a glycoform of a second protein. A secondary antibody or lectin is optionally labeled, e.g., with a fluorescent label or enzyme, or is configured to bind a label (e.g., is biotinylated). The composition can include reagents for amplifying a nucleic acid tag or tags (e.g., a polymerase, nucleotides, etc.), reagents for detecting a lectin or secondary antibody (e.g., a fluorogenic or colorimetric substrate), or the like.

Kits comprising one or more elements of the compositions are also features of the invention. For example, a kit can include an antibody as described above, and optionally also a lectin, a secondary antibody against the first antibody, a second antibody against a second glycoform of the first protein, a third antibody against a glycoform of a second protein, reagents for amplifying a nucleic acid tag or tags, reagents for detecting a lectin or secondary antibody, and/or the like, packaged in one or more containers. Typically, the kit includes instructions for using the components of the kit to diagnose, prognose or monitor sepsis, pancreatitis or pancreatic cancer.

Systems for performing the above correlations are also a feature of the invention. Typically, the system will include system instructions that correlate the levels of one or more markers of the invention with a particular diagnosis, prognosis, etc. The system instructions can compare detected information as to marker levels with a database that includes correlations between the markers and the relevant phenotypes. The system includes provisions for inputting sample-specific information regarding marker detection information, e.g., through an automated or user interface, and for comparing that information to the database.

The system can include one or more data acquisition modules for detecting one or more marker levels. These can include sample handlers (e.g., fluid handlers), robotics, microfluidic systems, protein purification modules, detectors, chromatography apparatus, a mass spectrometers, thermocyclers, or combinations thereof, e.g., for acquiring samples, diluting or aliquoting samples, purifying marker materials (e.g., proteins), detecting markers, and the like. The sample to be analyzed or a composition as noted above is optionally part of the system, or can be considered separate from it.

Optionally, system components for interfacing with a user are provided. For example, the systems can include a user viewable display for viewing an output of computer-implemented system instructions, user input devices (e.g., keyboards or pointing devices such as a mouse) for inputting user commands and activating the system, etc. Typically, the system of interest includes a computer, wherein the various computer-implemented system instructions are embodied in computer software, e.g., stored on computer readable media.

The person skilled in the art would be aware that any statistically significant difference in level from control would be determinant of a diagnosis, prognosis or response. The degree of difference in the level of one or more marker(s) in a sample from a subject from the level of that marker(s) in a control that would be indicative of a significant change or difference and be determinant of a diagnosis, prognosis or response would be well within the skill of the ordinary person to determine. For the avoidance of doubt, and in the interests of clarity, it is preferred that a significant change is one in which the determined level of marker(s) varies by more than 5, 10, 15 or 20% from that of the control marker(s).

Definitions

Unless defined otherwise, all technical and scientific terms used herein have the same meaning as commonly understood by one of ordinary skill in the art to which the invention pertains. The following definitions supplement those in the art and are directed to the current application and are not to be imputed to any related or unrelated case, e.g., to any commonly owned patent or application. Although any methods and materials similar or equivalent to those described herein can be used in the practice for testing of the present invention, the preferred materials and methods are described herein. Accordingly, the terminology used herein is for the purpose of describing particular embodiments only, and is not intended to be limiting.

As used in this specification and the appended claims, the singular forms “a,” “an” and “the” include plural referents unless the context clearly dictates otherwise. Thus, for example, reference to “a protein” includes a plurality of proteins; reference to “a cell” includes mixtures of cells, and the like.

An “amino acid sequence” is a polymer of amino acid residues (e.g., a protein) or a character string representing an amino acid polymer, depending on context.

A “polypeptide” or “protein” is a polymer comprising two or more amino acid residues. The polymer can additionally comprise non-amino acid elements such as labels, quenchers, blocking groups, or the like and can optionally comprise modifications such as glycosylation or the like. The amino acid residues of the polypeptide can be natural or non-natural and can be unsubstituted, unmodified, substituted or modified.

The term “glycoprotein” refers to an amino acid sequence and one or more oligosaccharide (glycan) structures associated with the amino acid sequence. A given glycoprotein can have one or more “glycoforms”. Each of the glycoforms of the particular glycoprotein has the same amino acid sequence; however, the glycan(s) associated with distinct glycoforms differ by at least one glycan.

The term “glycan” refers to a polysaccharide (a polymer comprising two or more monosaccharide residues). “Glycan” can also be used to refer to the carbohydrate portion of a glycoconjugate, such as a glycoprotein or glycolipid. Glycans can be homo- or heteropolymers of monosaccharide residues, and can be linear or branched. “N-linked” glycans are found attached to the R-group nitrogen of asparagine residues in proteins, while “O-linked” glycans are found attached to the R-group oxygen of serine or threonine residues.

The “GU value” (or “glucose unit value”) of a glycan indicates its degree of approximate size. The GU value expresses essentially the elution time of a particular glycan from a chromatography column. Since the elution time expressed in real time or volume can vary depending on the individual column, its age, etc., the column is first calibrated with a standard mixture of glycose oligomers.

The term “A3FG1” throughout the specification includes both naturally occurring A3FG1 and A3FG1 obtained by digesting glycans with sialidase, galactosidase and/or α1,2 fucosidase. Accordingly, the term “A3FG1 derived from digestion of SLe^{x}” is understood herein to include A3FG1 naturally present as well as A3FG1 derived from digestion of SLe^{x}.

“Acute-phase proteins” are proteins whose plasma concentrations increase (positive acute phase proteins) or decrease (negative acute phase proteins) in response to inflammation, e.g., by 25% or more.

The term “subject” refers to an animal, more preferably a mammal, and most preferably a human. Typically, the subject is known to have or suspected of having a disease, disorder, or condition of interest, e.g., a cancer or chronic inflammation.

The term “marker” refers to a molecule that is detectable in a biological sample obtained from a subject and that is indicative of a disease, disorder, or condition of interest (or a susceptibility to the disease, disorder, or condition) in the subject. Markers of particular interest in the invention include glycans and glycoproteins showing differences in glycosylation between a sample of from an individual with the disease, disorder, or condition and a healthy control.

A “control sample” can originate from a single individual not affected by a disease, disorder, or condition of interest (e.g., cancer or chronic inflammation) or be a sample pooled from more than one such individual.

In the context of the invention, the term “isolated” refers to a biological material, such as a protein, which is substantially free from components that normally accompany or interact with it in its naturally occurring environment. The isolated material optionally comprises material not found with the material in its natural environment, e.g., a cell. A protein isolated from a cell or from serum, for example, can be purified or partially purified from the cell or serum.

An “immunoassay” makes use of the specific binding of an antibody to its antigen to identify and/or quantify the antigen in a sample. An immunoassay can involve a single antibody or two or more antibodies (to a single antigen or a plurality of antigens).

A variety of additional terms are defined or otherwise characterized herein.

### BRIEF DESCRIPTION OF THE DRAWINGS

FIG. 1 shows normal-phase chromatograms of N-glycans released from a) serum proteins from a patient with sepsis on days 1, 2, 4 and 8 of the disease; b) serum proteins from a patient with acute pancreatitis on days 1, 2, 5 and 8 of the disease and c) serum proteins from a healthy control. Glycan structures that were identified in peaks 1-26 are shown in Table 1. Changes persistent throughout the studied period are marked with arrows. Enlarged rectangular on sample S1 demonstrates that all the peaks are clearly detectable, although not well seen on this scale.

FIG. 2 shows negative ion nano-ESI MS/MS spectra of six N-glycans from the Sepsis Day 8 sample. (a) Man₅GlcNAc₂ with a structure showing the main diagnostic cleavage sites. The branching pattern on the 3- and 6-antennae in this and the other oligomannose glycans is reflected by the masses of the D, [D-18]⁻ and ^{O,3}A_{R-2} ions. (b) Biantennary glycan Gal₂GlcNAc₄Man₃ (A2G2). The F-type ion at m/z 424 specifies the Gal-GlcNAc antenna and the C₁ ion at m/z 179 locates the galactose to the non-reducing terminus. The composition of the 6-antenna is given by the D and [D-18]⁻ ions at m/z 688 and 670 respectively. (c) Fucosylated-bisected biantennary glycan. The location of the fucose is revealed by the masses of the ^{2,4}A_{R}, B_{R} and ^{2,4}A_{R-1} ions at m/z 1681, 1621 and 1478 respectively (fucose is lost). The ion at m/z 670, corresponding to [D-221]⁻ is diagnostic for the presence of the bisecting GlcNAc. (d) Triantennary glycans Gal₃GlcNAc₅Man₃ (A3G3). Mixture of isomers. The E-type ion at m/z 831 and D/D-18]⁻ ions at m/z 688/670 are diagnostic for the isomer with a branched 3-antenna. D, [D-18]⁻ and [D-36]⁻ ions at m/z 1053, 1035 and 1017 respectively show the presence of the isomer with a branched 6-antenna. (e) Fucosylated triantennary glycans Gal₃GlcNAc₅Man₃Fuc₁ (A3G3F and FcA3G3). The presence of core- and antenna-fucosylation is reflected by two sets of ^{2,4}A_{R}, B_{R} and ^{2,4}A_{R-1} ions at m/z 1989.7, 1929.7 and 1786.6 and at m/z 1843.7, 1783.6 and 1640.6 respectively. The F-type ion at m/z 570 confirms antenna-fucosylation and the absence of a Gal-Fuc ion at m/z 325 shows fucose substitution on GlcNAc forming the Lewis X epitope. The E ion at m/z 977 shows that the antenna-fucose is substituted on the 3-antenna. (f) Tetra-antennary glycan Gal₄GlcNAc₆Man₃ (A4G4). The branching is reflected by the E (m/z 831) and D/[D-18]⁻/[D-36]⁻ ions at m/z 1053, 1035 and 1017 respectively.

FIG. 3 shows MALDI-TOF Mass spectra of desialylated glycans from the serum of the normal patient (a) and from the patients with sepsis (b) and pancreatitis (c) on Day 8. the spectra have been smoothed (Savitsky-Golay 2×2) and the peaks have been labelled with the masses of the peaks that are listed in Table 2.

FIG. 4 shows changes in trisialylated A3G3S3 and A3G3S3F structures. N-linked glycans were released from serum proteins and separated by WAX-HPLC according to the number of sialic acids. Trisialylated fractions were collected and analyzed by NP-HPLC as described in the Materials and Methods section.

Panel A—NP-HPLC profiles of trisialylated fractions of the patient with sepsis on day one (S1) and day eight (S8) of the disease, pancreatitis on day one (P1) and day eight (P8) of the disease and control serum (C).

Panel B—Changes in A3G3S3 and A3G3S3F structures during the first eight days of sepsis and pancreatitis.

FIG. 5 shows changes in tetrasialylated structures. N-linked glycans were released from serum proteins and separated by WAX-HPLC according to the number of sialic acids. Tetrasialylated fractions were collected and analyzed by NP-HPLC as described in the Materials and Methods section. Profiles of tetrasialylated glycan fractions of a patient with sepsis on day one (S1) and day eight (S8) of the disease, of a patient with pancreatitis on day one (P1) and day eight (P8) of the disease and control serum (C) are shown.

FIG. 6 shows changes in oligomannose structures. N-linked glycans were released from serum proteins and separated by WAX-HPLC. Neutral fractions were collected and analyzed by NP-HPLC as described in the Materials and Methods section.

Panel A—Profiles of neutral glycan fractions of a patient with sepsis on day one (S1) and day eight (S8) of the disease, of a patient with pancreatitis on day one (P1) and day eight (P8) of the disease and control serum (C) are shown. Peaks containing major oligomannose structures (Man6, Man7 and Mang) are indicated. The peak containing Man5 also contained FcA2B since it was partly resistant to Jack Bean mannosidase digestion (data not shown).

Panel B—Changes in oligomannose structures between day one (S1) and day eight (S8) of sepsis, and day one (P1) and day eight (P8) of pancreatitis. Mannose structures are given as % of neutral glycans.

FIG. 7. Changes in the degree of branching. N-linked glycans were released from serum proteins and treated with a combination of a sialidase from Arthrobacter ureafaciens (ABS), β-galactosidase from S. pneumoniae (SPG) and α-fucosidase from bovine kidney (BKF), which reduced glycans into basic mono-, bi-, tri- and tetraantennary structures.

Panel A—NP-HPLC profile of a glycan pool after ABS+SPG+BKF digestion. Structures of the main core glycans are shown above their corresponding peaks.

Panel B—Changes in the degree of branching between day one (S1) and eight (S8) of sepsis, and day one (P1) and eight (P8) of pancreatitis are shown as percentages of the total core structures.

### DETAILED DESCRIPTION OF THE INVENTION

As used herein, an “antibody” is a protein comprising one or more polypeptides substantially or partially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. A typical immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively. Antibodies exist as intact immunoglobulins or as a number of well-characterized fragments produced by digestion with various peptidases. Thus, for example, pepsin digests an antibody below the disulfide linkages in the hinge region to produce F(ab)′2, a dimer of Fab which itself is a light chain joined to VH-CH1 by a disulfide bond. The F(ab)′2 may be reduced under mild conditions to break the disulfide linkage in the hinge region thereby converting the (Fab′)2 dimer into a Fab′ monomer. The Fab′ monomer is essentially a Fab with part of the hinge region (see, Fundamental Immunology, W. E. Paul, ed., Raven Press, N.Y. (1999), for a more detailed description of other antibody fragments). While various antibody fragments are defined in terms of the digestion of an intact antibody, one of skill will appreciate that such Fab′ fragments may be synthesized de novo either chemically or by utilizing recombinant DNA methodology. Thus, the term antibody, as used herein, includes antibodies or fragments either produced by the modification of whole antibodies or synthesized de novo using recombinant DNA methodologies. Antibodies include, e.g., polyclonal and monoclonal antibodies, and multiple or single chain antibodies, including single chain Fv (sFv or scFv) antibodies in which a variable heavy and a variable light chain are joined together (directly or through a peptide linker) to form a continuous polypeptide, as well as humanized or chimeric antibodies.

Antibodies, e.g., antibodies specific for polypeptides bearing glycan markers of the invention, can be generated by methods well known in the art. Such antibodies can include, but are not limited to, polyclonal, monoclonal, chimeric, humanized, single chain, Fab fragments and fragments produced by a Fab expression library.

Polypeptides do not require biological activity for antibody production. However, the polypeptide or oligopeptide is antigenic. Peptides used to induce specific antibodies typically have an amino acid sequence of at least about 5 amino acids, and often at least 10 or 20 amino acids. Short stretches of a polypeptide can optionally be fused with another protein, such as keyhole limpet hemocyanin, and antibodies produced against the fusion protein or polypeptide.

Numerous methods for producing polyclonal and monoclonal antibodies are known to those of skill in the art, and can be adapted to produce antibodies specific for polypeptides bearing markers of the invention. See, e.g., Coligan (1991) Current Protocols in Immunology Wiley/Greene, NY; and Harlow and Lane (1989) Antibodies: A Laboratory Manual Cold Spring Harbor Press, NY; Stites et al. (eds.) Basic and Clinical Immunology (4th ed.) Lange Medical Publications, Los Altos, Calif., and references cited therein; Goding (1986) Monoclonal Antibodies: Principles and Practice (2d ed.) Academic Press, New York, N.Y.; Fundamental Immunology, e.g., 4th Edition (or later), W. E. Paul (ed.), Raven Press, N.Y. (1998); and Kohler and Milstein (1975) Nature 256: 495-497. Other suitable techniques for antibody preparation include selection of libraries of recombinant antibodies in phage or similar vectors. See, Huse et al. (1989) Science 246: 1275-1281; and Ward, et al. (1989) Nature 341: 544-546. Additional details on antibody production and engineering techniques can be found in U.S. Pat. No. 5,482,856, Borrebaeck (ed) (1995) Antibody Engineering, 2nd Edition Freeman and Company, NY (Borrebaeck); McCafferty et al. (1996) Antibody Engineering, A Practical Approach IRL at Oxford Press, Oxford, England (McCafferty), Paul (1995) Antibody Engineering Protocols Humana Press, Towata, N.J. (Paul), Ostberg et al. (1983) Hybridoma 2: 361-367, Ostberg, U.S. Pat. No. 4,634,664, and Engelman et al. U.S. Pat. No. 4,634,666. Specific monoclonal and polyclonal antibodies and antisera will usually bind with a KD of at least about 0.1 μM, preferably at least about 0.01 μM or better, and most typically and preferably, 0.001 μM or better.

Molecular Biological Techniques

In practicing the present invention, many conventional techniques in molecular biology, microbiology, and recombinant DNA technology are optionally used. These techniques are well known and are explained in, for example, Berger and Kimmel, Guide to Molecular Cloning Techniques, Methods in Enzymology volume 152 Academic Press, Inc., San Diego, Calif.; Sambrook et al., Molecular Cloning—A Laboratory Manual (3rd Ed.), Vol. 1-3, Cold Spring Harbor Laboratory, Cold Spring Harbor, N.Y., 2000 and Current Protocols in Molecular Biology, F. M. Ausubel et al., eds., Current Protocols, a joint venture between Greene Publishing Associates, Inc. and John Wiley & Sons, Inc., (supplemented through 2007). Other useful references, e.g. for cell isolation and culture include Freshney (1994) Culture of Animal Cells, a Manual of Basic Technique, third edition, Wiley-Liss, New York and the references cited therein; Payne et al. (1992) Plant Cell and Tissue Culture in Liquid Systems John Wiley & Sons, Inc. New York, N.Y.; Gamborg and Phillips (Eds.) (1995) Plant Cell, Tissue and Organ Culture; Fundamental Methods Springer Lab Manual, Springer-Verlag (Berlin Heidelberg New York) and Atlas and Parks (Eds.) The Handbook of Microbiological Media (1993) CRC Press, Boca Raton, Fla. Methods of making nucleic acids (e.g., by in vitro amplification, purification from cells, or chemical synthesis), methods for manipulating nucleic acids (e.g., site-directed mutagenesis, by restriction enzyme digestion, ligation, etc.), and various vectors, cell lines and the like useful in manipulating and making nucleic acids are described in the above references. In addition, essentially any polynucleotide can be custom or standard ordered from any of a variety of commercial sources.

In addition to other references noted herein, a variety of purification/protein purification methods are well known in the art, including, e.g., those set forth in R. Scopes, Protein Purification, Springer-Verlag, N.Y. (1982); Deutscher, Methods in Enzymology Vol. 182: Guide to Protein Purification, Academic Press, Inc. N.Y. (1990); Sandana (1997) Bioseparation of Proteins, Academic Press, Inc.; Bollag et al. (1996) Protein Methods, 2nd Edition Wiley-Liss, NY; Walker (1996) The Protein Protocols Handbook Humana Press, NJ; Harris and Angal (1990) Protein Purification Applications: A Practical Approach IRL Press at Oxford, Oxford, England; Harris and Angal Protein Purification Methods: A Practical Approach IRL Press at Oxford, Oxford, England; Scopes (1993) Protein Purification: Principles and Practice 3rd Edition Springer Verlag, NY; Janson and Ryden (1998) Protein Purification: Principles, High Resolution Methods and Applications, Second Edition Wiley-VCH, NY; and Walker (1998) Protein Protocols on CD-ROM Humana Press, NJ; and the references cited therein.

### Examples

It is understood that the examples and embodiments described herein are for illustrative purposes only and that various modifications or changes in light thereof will be suggested to persons skilled in the art and are to be included within the spirit and purview of this application and scope of the appended claims. Accordingly, the following examples are offered to illustrate, but not to limit, the claimed invention.

Materials and Methods

Serum Samples

Serum samples were collected at the University hospital Zagreb. Sera from a septic patient and a patient with acute pancreatitis were taken at the time of reporting to hospital and then three more times through the first eight days of hospitalization. Patients claimed to report to hospital on the first day of feeling sick, so this was assumed to be the first day of disease. The blood from a healthy individual, matched by sex and age, was drawn on one occasion only. The possibility of any inflammatory condition in the control subject was additionally excluded by measuring C-reactive protein (CRP was lower than 0.5 mg/dL). The enrolled patients were individuals who fulfilled clinical criteria of sepsis or acute pancreatitis and who had signed the informed consent to participate. This study conformed to the ethical guidelines of the 1975 Declaration of Helsinki and was approved by the Institutional Review Boards of the University hospital center Zagreb and the University of Zagreb Faculty of Pharmacy and Biochemistry.

Glycan Release and Labelling

The N-glycans from 10 μL of sera were analyzed as described previously (Royle et al manuscript submitted). Briefly, the proteins from sera were immobilized in a block of SDS-polyacrylamide gel and N-glycans were released by digestion with recombinant N-glycosidase F (PNGase F, Roche Diagnostics). After extraction, glycans were fluorescently labeled with 2-aminobenzamide (LudgerTag 2-AB labeling kit Ludger Ltd., Abingdon, UK).

Normal Phase (NP)-HPLC

Released glycans were then subjected to normal-phase high performance liquid chromatography (NP-HPLC) on a TSK Amide-80 250×4.6 mm column (Anachem, Luton, UK) at 30° C. with 50 mM formic acid adjusted to pH 4.4 with ammonia solution as solvent A and acetonitrile as solvent B. 180 and 120 min runs were on a 2695 Alliance separations module (Waters, Milford, Mass.). HPLCs were equipped with a Waters temperature control module and a Waters 2475 fluorescence detector set with excitation and emission wavelengths of 330 and 420 nm. The system was calibrated using an external standard of hydrolyzed and 2-AB-labeled glucose oligomers from which the retention times for the individual glycans were converted to glucose units (GU) (Royle, L., Radcliffe, C. M., et al. 2006). Glycans were analyzed on the basis of their elution positions and measured in glucose units then compared to reference values in the “Glycobase” database (available at: http://glycobase.ucd.ie/cgi-bin/glycobase.cgi) for preliminary structure assignment (Royle et al submitted).

Weak Anion Exchange (WAX)-HPLC

Glycans were separated according to the number of sialic acids by weak anion exchange HPLC. The analysis was performed using a Vydac 301VHP575 7.5×50-mm column (Anachem Ltd., Luton, Bedfordshire, UK) (Royle, L., Radcliffe, C. M., et al. 2006). Compounds were retained on the column according to their charge density, the higher charged compounds being retained the longest. Separated fractions were collected and subjected to NP-HPLC.

Exoglvcosidase Digestions

Glycans, both from total glycan pool and WAX separated fractions, were sequenced by exoglycosidase digestions followed by NP-HPLC (Royle, L., Radcliffe, C. M., et al. 2006). The exoglycosidase digestions of 2-AB labeled glycans were carried out with the following enzymes obtained from Prozyme, San Leandro, Calif., USA: ABS, Arthrobacter ureafaciens sialidase (Glyko Sialidase A,) specific for α2-3,6,8 sialic acids; NAN1, Streptococcus. pneumoniae neuraminidase—(Sialidase S) releases α2-3,8 linked sialic acid; BTG, Bovine testes β-galactosidase specific for β1-3,4 & 6 linked galactose; SPG, S. pneumoniae β-galactosidase, specific for β1-4-linked galactose; BKF, bovine kidney α-fucosidase digests α1-2,6>>3,4-fucose; GUH, β-N-acetyl-glucosaminidase digests N-acetylglucosamine but not the bisect; JBM, Jack Bean α-mannosidase, AMF, Almond meal α-fucosidase-removes α1-314-fucose; XMF, Xanthomonus sp. α1-2-fucosidase (New England Biolabs (Hitchin, Herts, UK). Samples were incubated overnight at 37° C. in 50 mM sodium acetate buffer, pH 5.5. except JBM digestion which were in 100 mM sodium acetate, 2 mM Zn2+, pH 5.0.

Mass Spectrometry

MALDI-TOF Mass Spectrometry

Desialylated glycan samples (1 μL of an aqueous solution) were cleaned with a Nafion 117 membrane (Börnsen, K. O., Mohr, M. D., Widmer, H. M. 1995) before analysis with 2,5-dihydroxybenzoic acid (DHB) on a Waters-Micromass (Manchester, UK) TofSpec 2E reflectron-TOF mass spectrometry operated in reflectron mode with delayed extraction (Harvey, D. J. 1993)

Nano-Electrospray Mass Spectrometry

Nano-electrospray mass spectrometry was performed with a Waters-Micromass quadrupole-time-of-flight (Q-Tof) Ultima Global instrument. Samples of non-2AB labelled glycans in 1:1 (v:v) methanol:water containing 0.5 mM ammonium phosphate were infused through Proxeon (Proxeon Biosystems, Odense, Denmark) nanospray capillaries as detailed in Harvey, D. J., 2005.

Results

The major N-linked glycans structures from the serum glycoproteins of patients with sepsis or acute pancreatitis were identified and quantified during the first eight days of the disease, and compared to glycans present on normal serum glycoproteins.

Glycan Profiles of the Whole Serum

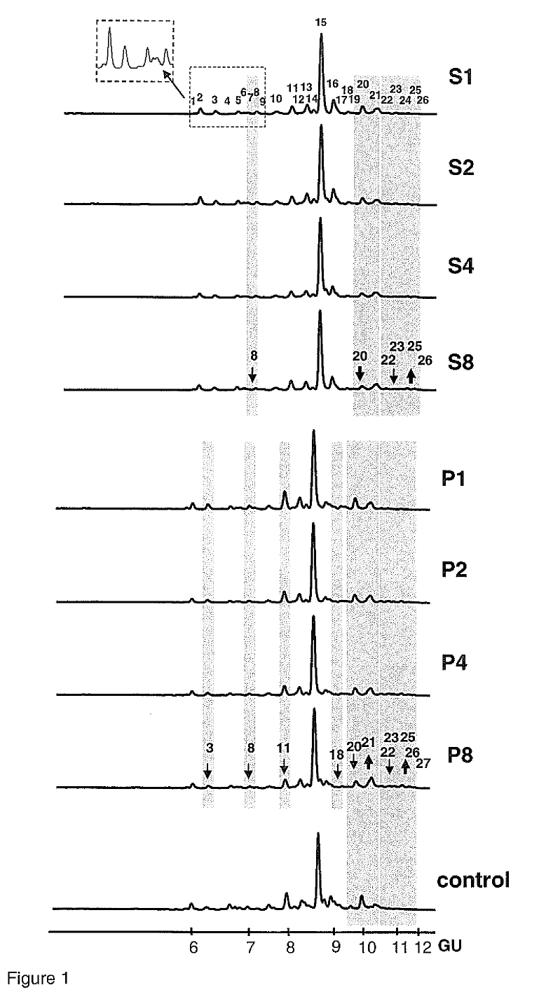

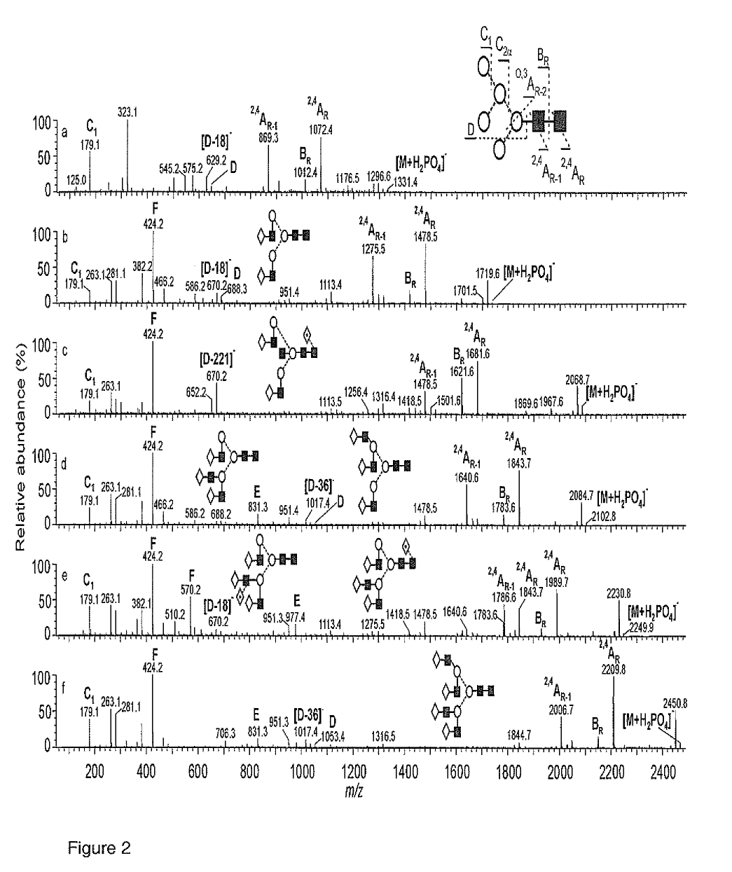

N-linked glycans were released from sequentially taken sera of a septic patient and a patient with acute pancreatitis as well as from a healthy individual. NP-HPLC profiles of patients through the first eight days of disease, as well as the glycan profile from the healthy individual are shown in FIG. 1. Structures of the glycans in each peak were identified by combinations of exoglycosidase digestions, chromatographic (NP-HPLC and WAX-HPLC) and mass spectrometric techniques. The NP-HPLC and WAX-HPLC in combination with exoglycosidase digestions enabled identification and quantification of the intact (sialylated) compounds, including those which coelute in the undigested profile. Quantifications of structures in chromatographic data are expressed as % of all integrated peaks. Mass spectrometric techniques, which were performed on desialylated and unlabelled glycans, complemented the chromatographic data and provided details such as the branching pattern of the triantennary glycans (see legend to FIG. 2 for details). Glycans in the major peaks from the HPLC profiles, identified by combination of all techniques mentioned, are listed in Table 1 together with the relative percentages. These structures are consistent with those already reported in healthy individuals (Royle et al submitted).

TABLE 1
NPHPLC data for N-linked glycans released from sera of patients
with sepsis and acute pancreatitis.

| | | | Sepsis | | | | Pancreatitis | | | | Control |
|---|---|---|---|---|---|---|---|---|---|---|---|
| | | | S1 | S2 | S5 | S8 | P1 | P2 | P4 | P8 | C |
| Peak | Structure | GU | % Area | | | | | | | | |
| 1 | A2B | 5.8 | 0.3 | 0.3 | 0.3 | 0.2 | 0.7 | 0.3 | 0.3 | 0.3 | 0.4 |
| 2 | FA2 | 5.9 | 2.9 | 3.8 | 2.3 | 2.7 | 3.0 | 2.0 | 2.2 | 2.3 | 2.7 |
| 3 | FA2B | 6.2 | 1.7 | 2.0 | 1.4 | 1.5 | 2.5 | 1.6 | 1.5 | 1.2 | 1.4 |
| | M5 | | | | | | | | | | |
| | A2[6]G1 | | | | | | | | | | |
| | A2B[6]G1 | | | | | | | | | | |
| 4 | A2[3]G1 | 6.5 | 0.1 | <0.1 | <0.1 | 0.1 | f | f | f | f | 0.2 |
| 5 | FA2[6]G1 | 6.6 | 1.5 | 1.8 | 1.2 | 1.8 | 1.3 | 0.9 | 1.1 | 1.2 | 2.2 |
| | A2B[3]G1 | | | | | | | | | | |
| 6 | FA2[3]G1 | 6.8 | 0.6 | 0.8 | 0.8 | 1.8 | 0.6 | 0.6 | 1.3 | 0.7 | 1.1 |
| | FA2B[6]G1 | | | | | | | | | | |
| 7 | FA2B[3]G1 | 6.9 | 1.1 | 1.1 | 0.7 | f | 0.9 | 0.5 | | 0.6 | 0.9 |
| 8 | M6 | 7.1 | 1.8 | 1.6 | 1.1 | 1.1 | 1.9 | 1.2 | 1.1 | 1.0 | 1.5 |
| | A2G2 | | | | | | | | | | |
| 9 | A2G1S(6)1 | 7.2 | 0.2 | 0.3 | 0.3 | 0.6 | 1.0 | 0.5 | 0.4 | 0.5 | 0.7 |
| | A2G1S(3)1 | | | | | | | | | | |
| 10 | FA2G2 | 7.6 | 2.6 | 2.7 | 1.9 | 2.4 | 2.6 | 2.0 | 2.0 | 1.8 | 3.3 |
| | FA2G1S1 | | | | | | | | | | |
| | A2BG1S1 | | | | | | | | | | |
| | FA2BG1S1 | | | | | | | | | | |
| | FA2BG2 | 7.7 | | | | | | | | | |
| 11 | M7 | 8.0 | 4.9 | 4.8 | 4.0 | 5.8 | 9.6 | 7.0 | 6.1 | 4.7 | 7.8 |
| | A2G2S1 | | | | | | | | | | |
| 12 | A2BG2S1 | 8.2 | 0.6 | f | f | 0.5 | 0.8 | 0.4 | 0.5 | 0.6 | 1.7 |
| 13 | FA2G2S1 | 8.4 | 6.0 | 7.4 | 5.6 | 5.3 | 6.8 | 6.0 | 5.8 | 5.6 | 6.2 |
| 14 | FA2BG2S1 | 8.5 | 2.2 | 3.1 | 1.8 | 1.3 | 2.1 | 1.4 | 1.6 | 2.1 | 2.5 |
| 15 | M8 | 8.7 | 44.2 | 42.0 | 50.4 | 44.9 | 40.3 | 46.6 | 46.3 | 43.9 | 36.9 |
| | A2G2S(3,3)2 | | | | | | | | | | |
| | A2G2S(3,6)2 | | | | | | | | | | |
| | A2G2S(6,6)2 | | | | | | | | | | |
| | A2FG2S(3)1 | | | | | | | | | | |
| | A2FG2S(6)1 | | | | | | | | | | |
| 16 | FA2G2S(3,6)2 | 9.1 | 10.9 | 11.3 | 9.8 | 10.1 | 7.8 | 6.7 | 6.8 | 7.0 | 12.6 |
| | FA2G2S(6,6)2 | | | | | | | | | | |
| | A2BG2S2 | | | | | | | | | | |
| | FA2BG2S2 | | | | | | | | | | |
| 17 | A3G3S2 | 9.4 | | | | 0.6 | | | | | |
| 18 | M9 | 9.5 | 1.5 | 1.8 | 1.3 | 1.2 | 1.7 | 1.5 | 1.5 | 1.3 | 2.1 |
| 19 | A2FG2S2 | 9.7 | 0.9 | 0.8 | 1.0 | 0.9 | 1.7 | 1.3 | 1.3 | 1.3 | |
| 20 | A3G3S3 | 10.0 | 5.4 | 4.6 | 3.9 | 3.4 | 6.5 | 5.9 | 5.6 | 5.5 | 8.0 |
| | A3G3S(3,3,3)3 | | | | | | | | | | |
| | A3G3S(6,3,3)3 | | | | | | | | | | |
| | A3G3S(6,6,6)3 | | | | | | | | | | |
| 21 | A3FG3S(3,3,6)3 | 10.5 | 6.6 | 5.5 | 6.8 | 6.8 | 6.0 | 7.4 | 7.5 | 9.7 | 6.0 |
| | A3FG3S(3,6,6)3 | | | | | | | | | | |
| | A3FG3S(6,6,6)3 | | | | | | | | | | |
| 22 | A4FG4S2 | 10.8 | 1.3 | 1.3 | 1.1 | 1.5 | 1.0 | 1.5 | 1.6 | 1.7 | 0.7 |
| 23 | A4G4S(6)4 | 11.1 | 1.1 | 1.1 | 1.1 | 1.1 | 0.7 | 1.5 | 1.6 | 1.7 | 0.6 |
| 24 | FA4G4S4 | 11.3 | 0.7 | 0.6 | 1.0 | 1.1 | 0.4 | 1.1 | 1.2 | 1.6 | 0.3 |
| 25 | A4FG4S4 | 11.6 | 0.7 | 0.7 | 1.0 | 1.3 | 0.3 | 1.2 | 1.5 | 2.0 | 0.2 |
| | A4G4S4Lac | | | | | | | | | | |
| 26 | A4F2G4S4 | 11.9 | 0.5 | 0.5 | 1.3 | 1.6 | f | 0.8 | 0.9 | 1.3 | 0.1 |
| | FA4FG4S4 | | | | | | | | | | |
| 27 | A4G4Lac2 | 12.1 | | | | | f | f | 0.4 | 0.5 | |

Major glycan structures present in the untreated glycan pool released from sera. (f = found, but in either very small amount or can not be differentiated (exact area value) from other peak).

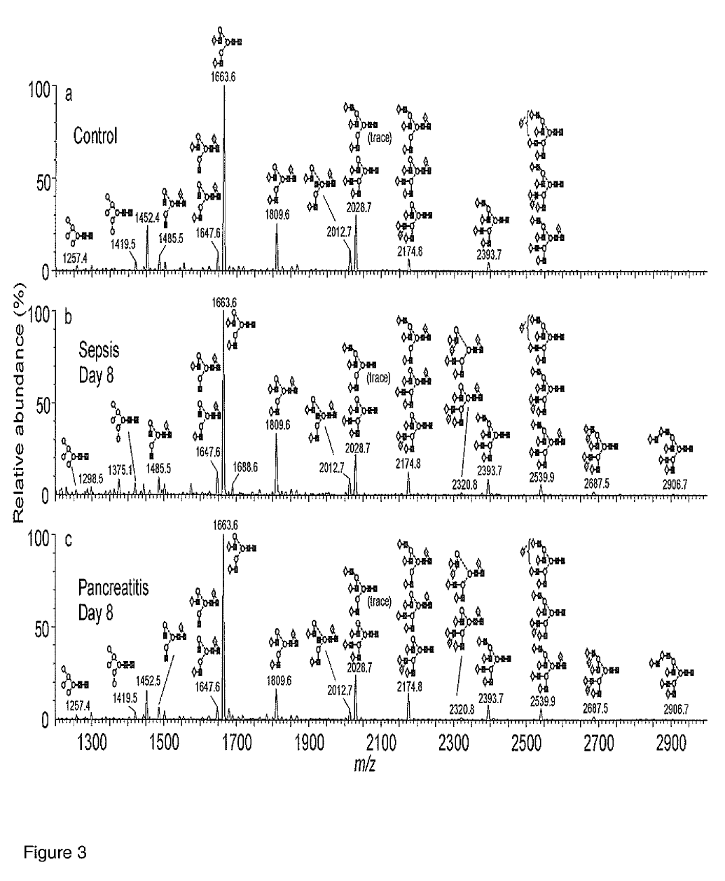

Table 2 lists the structures of the glycans that were identified by MS techniques and FIG. 3 shows the MALDI profiles of the desialylated glycans taken on Day 8 of disease compared with that from the control patient.

TABLE 2
Compositions, masses (monoisotopic unless stated otherwise)
and structures for the disialylated N-glycans as determined by MALDI-TOF
and ESI-MS.

| m/z | | Composition | | | | |
|---|---|---|---|---|---|---|
| ESI | MALDI | | | | | |
| (M + H2PO4)⁻) | ([M + Na]⁺) | Hex | HNAc | Fuc | Structure | Code |
| 1257.4 | 1331.4 | 5 | 2 | 0 | 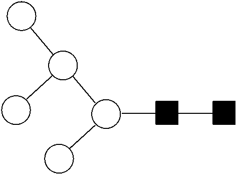 | M5 |
| 1282.4 | 1356.4 | 3 | 3 | 1 | 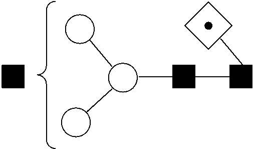 | FA1 |
| 1298.5 | 1372.4 | 4 | 3 | 0 | 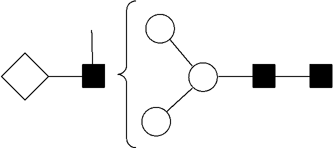 | A1G1 |
| 1339.5 | 1413.4 | 3 | 4 | 0 | 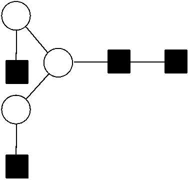 | A2 |
| 1419.5 | 1493.5 | 6 | 2 | 0 | 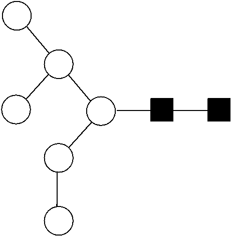 | M6 |
| 1460.5 | 1534.5 | 5 | 3 | 0 | 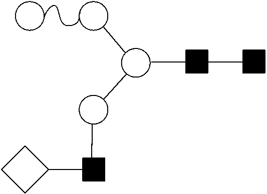 | A1G1M4 |
| 1485.5 | 1559.5 | 3 | 4 | 1 | 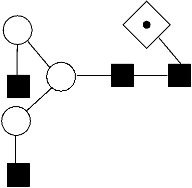 | FA2 |
| 1501.5 | 1575.5 | 4 | 4 | 0 | 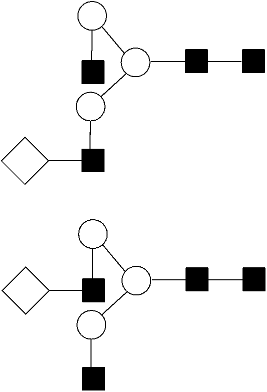 | A2G1 |
| 1542.5 | 1616.5 | 3 | 5 | 0 | 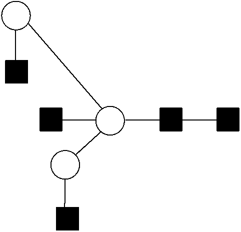 | A2B |
| 1581.5 | 1655.6 | 7 | 2 | 0 | 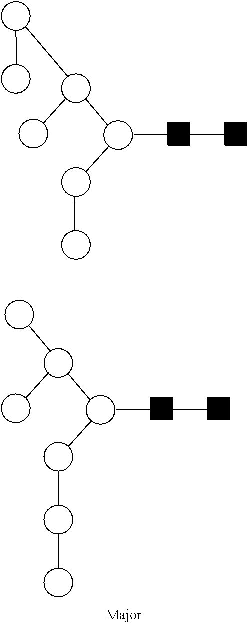 | M7 |
| 1622.5 | 1696.5 | 6 | 3 | 0 | 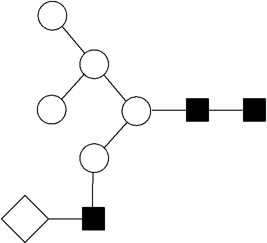 | A1G1M5 |
| 1647.6 | 1721.6 | 4 | 4 | 1 | 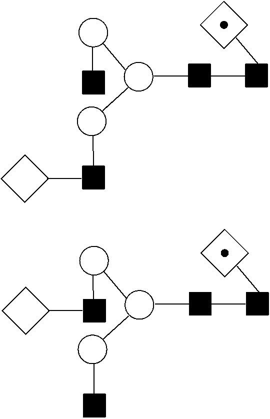 | FA2G1 |
| 1663.6 | 1737.6 | 5 | 4 | 0 | 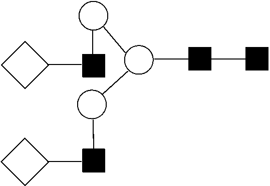 | A2G2 |
| 1688.6 | 1762.6 | 3 | 5 | 1 | 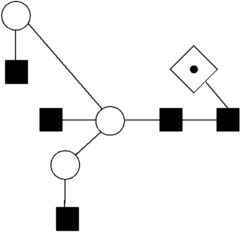 | FA2B |
| 1704.6 | 1778.6 | 4 | 5 | 0 | 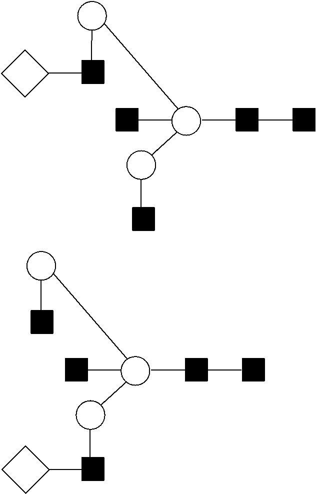 | A2BG1 |
| 1743.6 | 1817.7 | 8 | 2 | 0 | 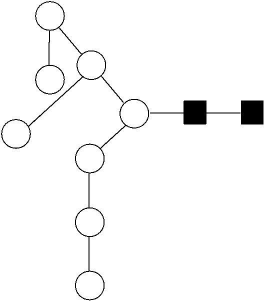 | M8 |
| 1809.6 | 1883.6 | 5 | 4 | 1 | 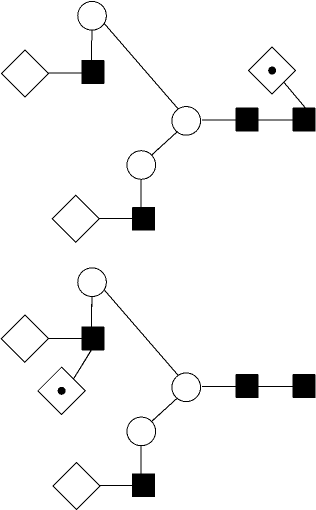 | FA2G2 A2FG2 |
| 1850.7 | 1924.6 | 4 | 5 | 1 | 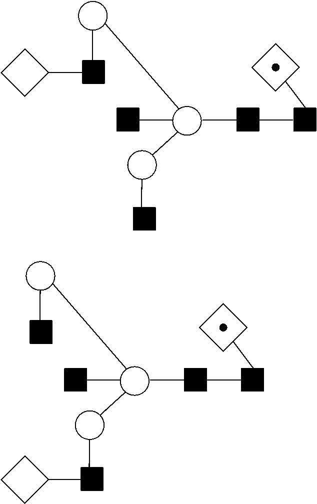 | FA2BG1 |
| 1866.7 | 1940.6 | 5 | 5 | 0 | 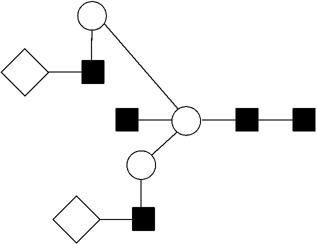 | A2BG2 |
| 1905.6 | 1979.6 | 9 | 2 | 0 | 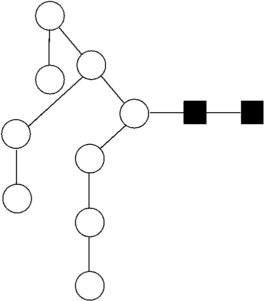 | M9 |
| 1955.7 | 2029.7 | 5 | 4 | 2 | 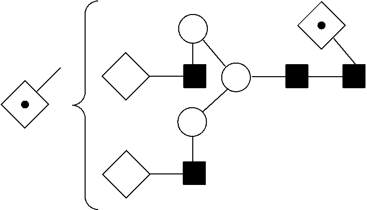 | FA2FG2 |
| 2012.7 | 2086.7 | 5 | 5 | 1 | 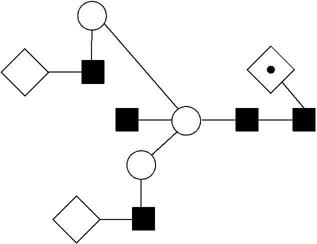 | FA2BG2 |
| 2028.7 | 2102.7 | 6 | 5 | 0 | 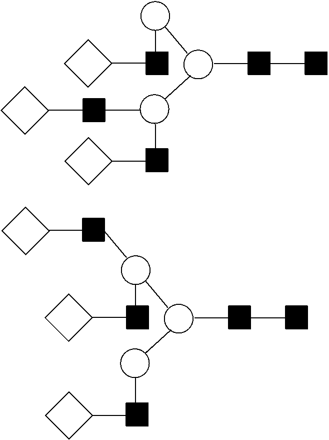 | A3G3 |
| 2174.8 | 2248.8 | 6 | 5 | 1 | 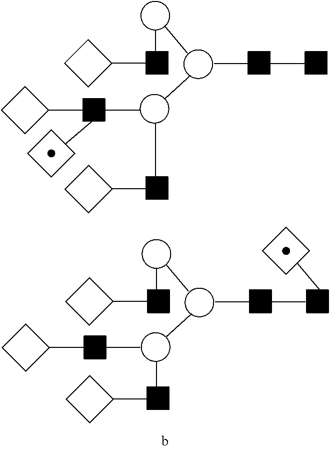 | A3FG3 FA3G3 |
| 2320.8 | 2394.8 | 6 | 5 | 2 | 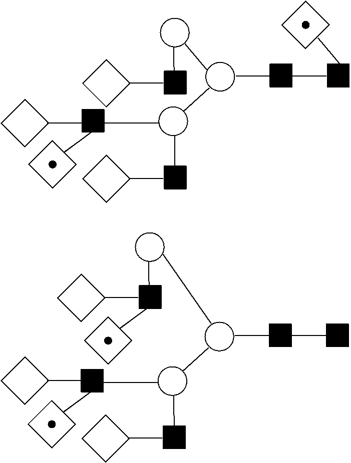 | FA3FG3 A3F2G3 |
| 2393.7 | 2467.8 | 7 | 6 | 0 | 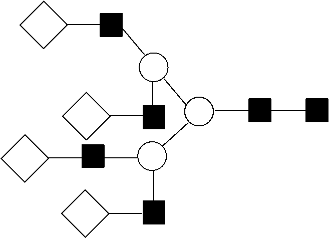 | A4G4 |
| 2539.9 | 2613.8 | 7 | 6 | 1 | 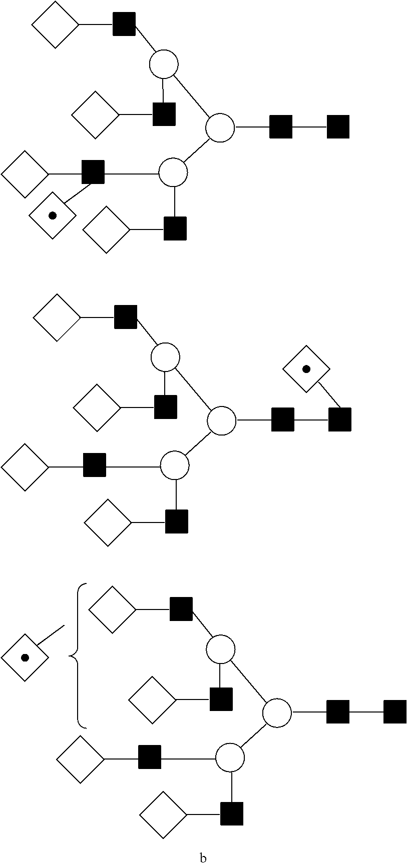 | A4FG4 FA4G4 |
| 2687.5^{a} | 2759.9 | 7 | 6 | 2 | 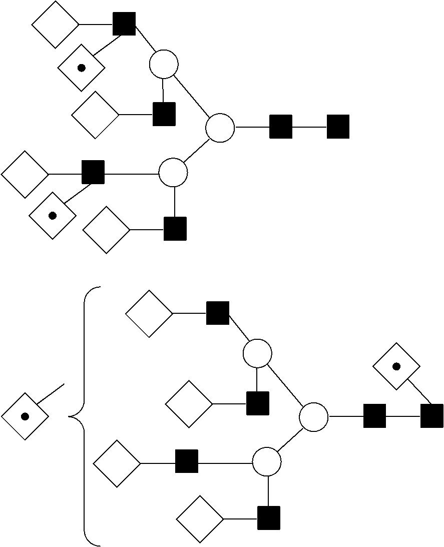 | A4F2G4 FA4FG4 |
| 2760.5^{a} | 2833.0 | 8 | 7 | 0 | 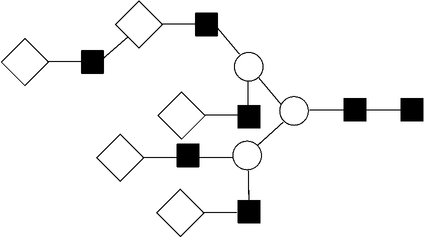 | A4G4Lac |
| 2833.6^{a} | 2906.0 | 7 | 6 | 3 | 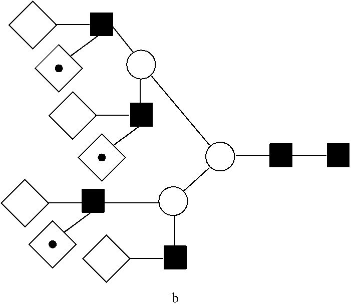 | A4F3G4 |
| 2906.7^{a} | 2979.0 | 8 | 7 | 1 | 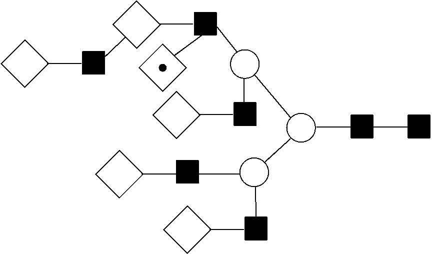 | A4FG4Lac |
| 3125.8^{a} | 3199.9 | 9 | 8 | 0 | | Polylac? |
| 3272.0^{a} | 3345.9 | 9 | 8 | 1 | | Polylac? |

Measured masses were within 0.1 mass units for the ESI spectra and 0.3 mass units for MALDI-TOF. Symbol representation of glycan structure is: GlcNAc, black square; galactose, white diamond; fucose, diamond with a dot inside; sialic acid, black star; βlinkage, solid line; alinkage, dotted line; 1-6 linkage, \; 1-4 linkage, −; 1-3 linkage, /; 1-2 linkage, |. (a. Average mass; b. Position of fucose on 3-antenna based on known structure of α1-acid glycoprotein.)

The analysis of the glycan profiles from patients with acute pancreatitis and sepsis identified several deviations from the healthy profile, as well as changes that occurred during the course of the disease. The most obvious and persistent changes during the first eight days of both conditions were changes in peaks numbered 20 to 26 (FIG. 1), identified as being trisialylated and tetrasialylated structures with and without sialyl Lewis x.

Changes in the Sialyl Lewis X Structures

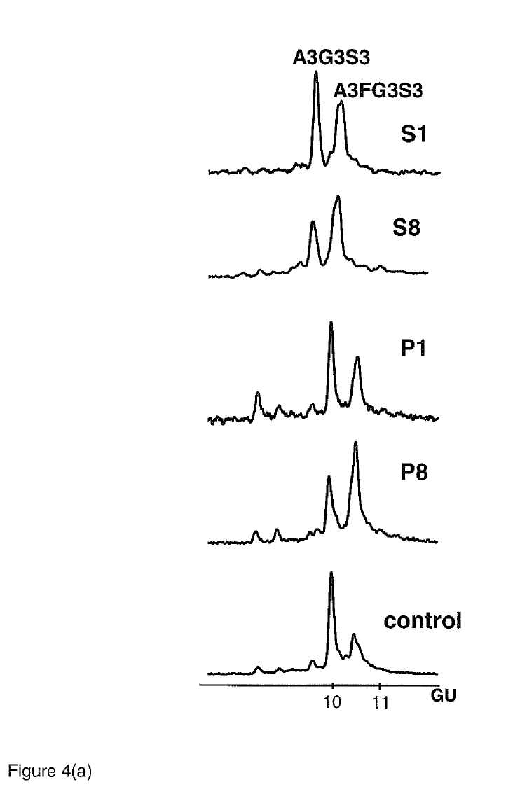

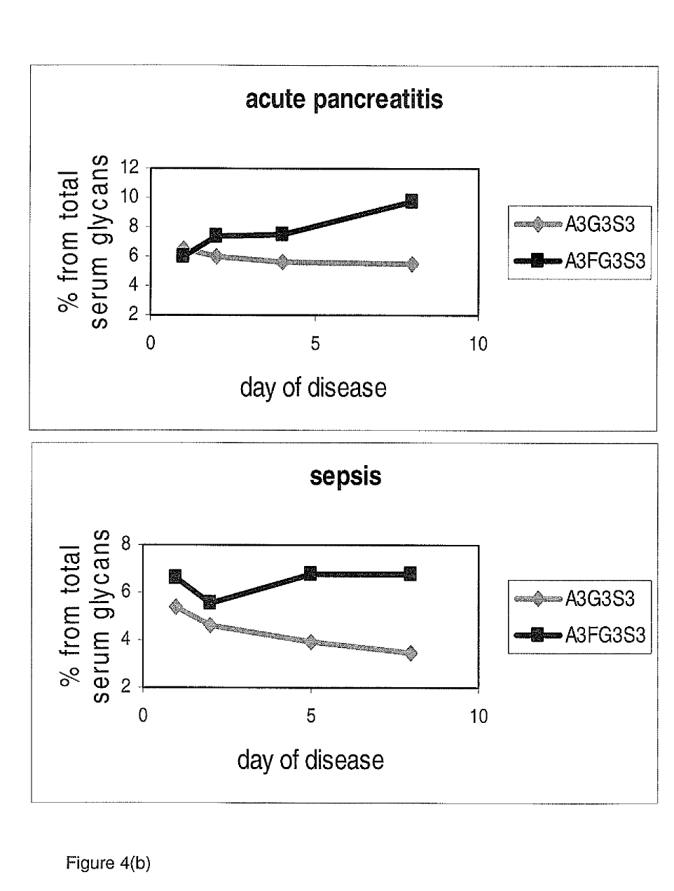

Changes in trisialylated structures in glycan profiles were persistent in both diseases throughout the studied period. NP-HPLC analysis of trisialylated glycans isolated by WAX-HPLC (FIG. 4) showed that the amount of the trisialylated glycan A3G3S3, decreased relative to the amount of the fucosylated A3FG3S3 (sialyl Lewis X) during the period studied, particularly in sepsis. The non fucosylated form decreased in sepsis from 5.4 to 3.4% in the total glycan pool and in pancreatitis it decreases from 6.5 to 5.5%, whilst in the healthy control this fraction represented 8% of total glycan pool. The fucosylated sialyl Lews X form increased in sepsis from 6.6 to 6.8%, while in pancretitis this increase was more expressed at 6.0 to 9.7% of the total glycan pool,. compared to 6% in the control (see Table 1).

HPLC and exoglycosidase sequencing showed that the A3FG3S3 structure digested with almond meal α-fucosidase (AMF) which removes fucose α1-3 or α1-4 linked to GlcNAc and that the galactose on this GlcNAc was removed by β1-4 galactosidase indicating that this was a Lewis X rather than a Lewis A structure. The outer-arm fucosylation was also found by negative ion MS/MS analysis (FIG. 2e) on desialylated monofucosylated triantennary glycans. The location of the none core fucose was shown to be on a GlcNAc residue by the absence of a C₁ ion corresponding to Gal-Fuc in the MS/MS analysis.

Negative ion MS/MS showed the presence of both core and outer arm fucosylated structures. The relative percentage of the core to antenna-mono-fucosylated triantennary glycans, as measured by the ratio of the ^{2,4}A_{R} ions in the negative ion MS/MS spectrum was 34% antenna and 66% core of the control sample, whereas in the sepsis Day 1 and Day 8 samples, the amount of antenna-fucosylated glycans rose to 52% and 63% for the two days respectively. In pancreatitis, the relative amount of antenna-fucosylated to core fucosylated triantennary glycan rose to 78% on Day 2 and 85% on Day 8 This shows a clear increase in the Lewis X structure relative to the core fucosylated structure with time in both diseases.

Changes in Tetrasialvlated Structures

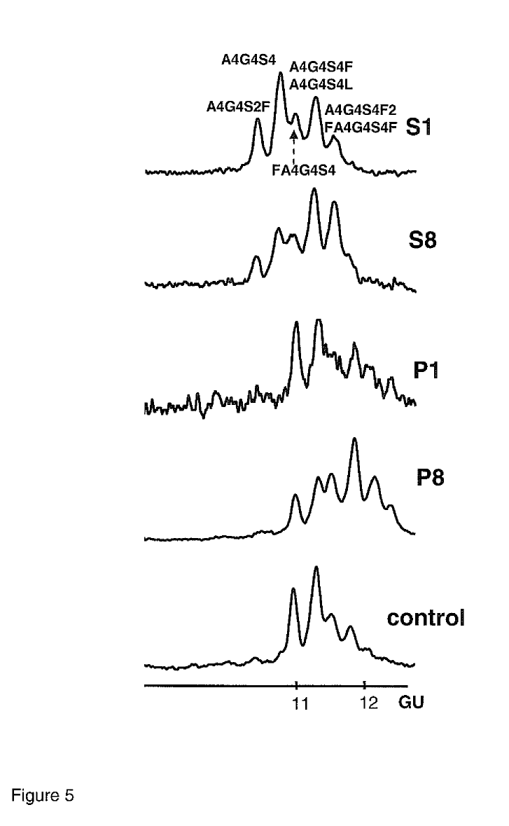

From the HPLC profiles shown in FIG. 1 it is evident that the tetrasialylated structures were elevated compared to the control profile and that the amount of these structures in the whole glycan pool changed during the course of both diseases. Changes observed were additionally analyzed by NP-HPLC analysis of the tetrasialylated fraction obtained from WAX HPLC which revealed an increase in outer-arm fucosylated structures over non-fucosylated ones (FIG. 5).

Negative ion MS/MS of the desialylated monofucosylated tetra-antennary showed that 54% of the fucose was outer arm in the control sample compared to sepsis samples at Day 1 (75%) and Day 8 (86%) and 92% in both of the pancreatitis samples. This also suggests a move to the Lewis X structure on tetra-antennary structures during the course of disease. The presence of outer arm fucosylation on di-sialylated biantennary structures was difficult to measure quantitatively due to it low abundance. However negative ion MS/MS (ratio of the ^{2,4}A_{R} ions) analysis of the disialylated samples detected a trace of the outer-arm-fucosylated biantennary glycan present in the control and sepsis Day 1 sample (about 2% of the fucosylated biantennary structures) which rose to about 7% on sepsis Day 8. The amount of antenna-fucosylated biantennary glycans in the pancreatitis samples appeared to be somewhat higher, reaching approximately 10% on Days 2 and 8. The fragmentation pattern showed that the fucose was mainly substituted in the 6-antenna (shifts in the D and [D-18]⁻ ions.

Changes in Total Fucose

Changes in fucose levels through the course of disease were also seen when we analyzed all fucose-containing glycans after exoglycosidase digestions (ABS+SPG) or (ABS+SPG+AMF) of whole glycan pools. Both conditions showed increase in the outer arm fucosylation (sepsis ˜50% difference between Day 1 and Day 8, pancreatitis ˜30% increase), while core fucosylation decreased in both conditions (˜15%).

Changes in Oligomannose Structures

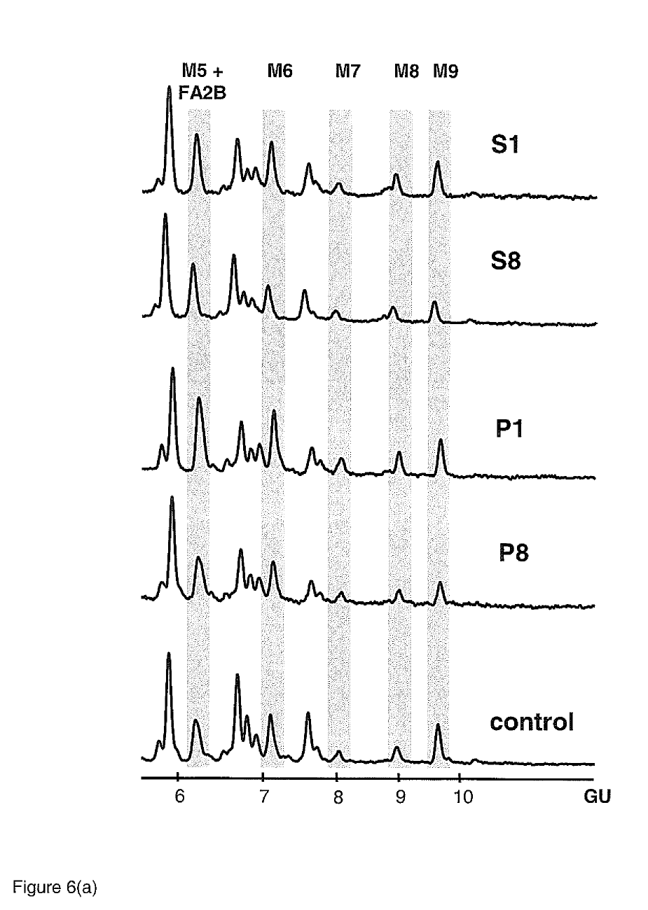

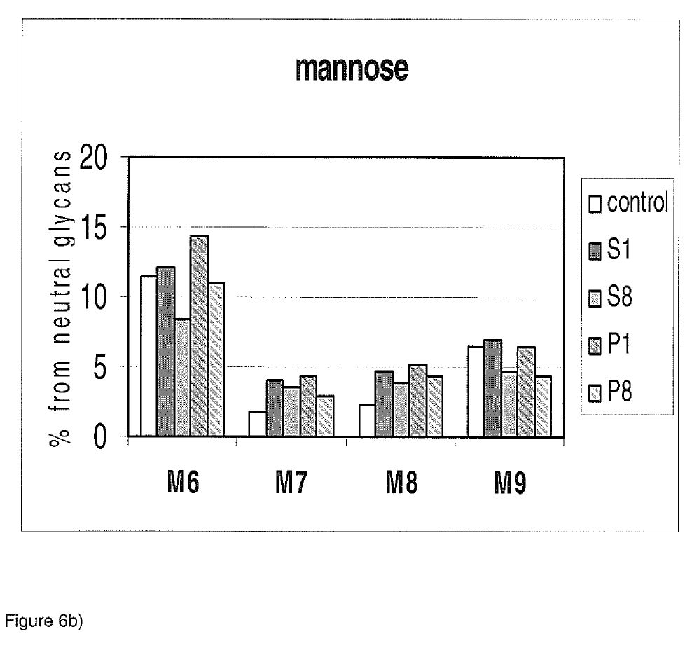

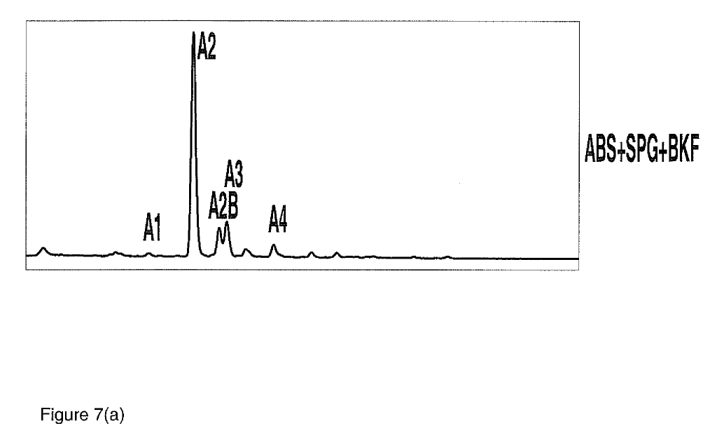

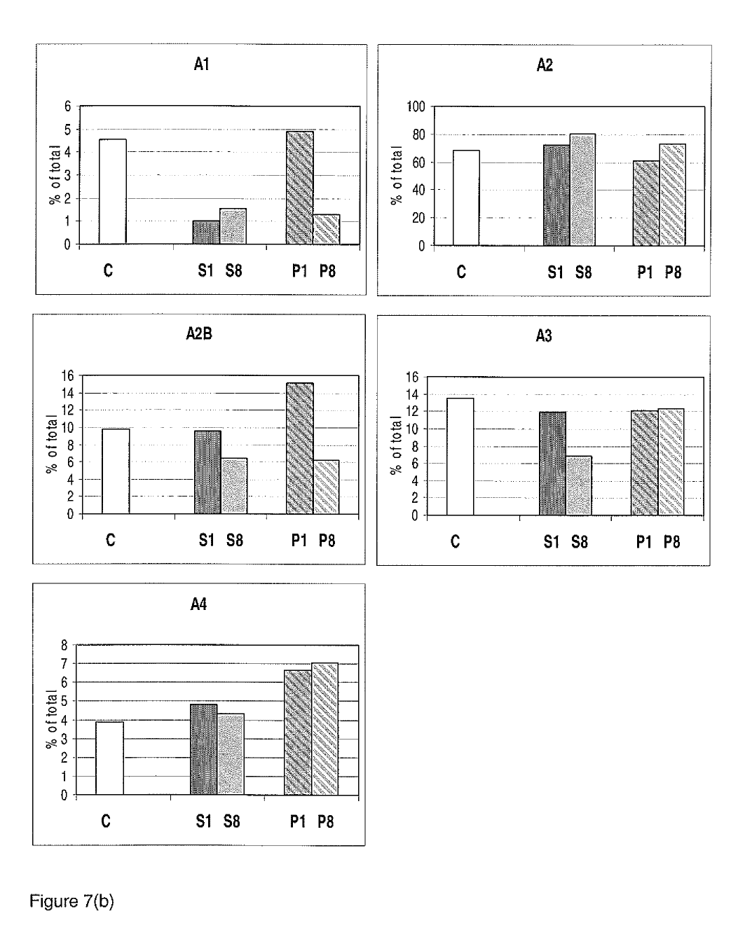

NP-HPLC profile of neutral fractions (prepared by WAX HPLC) showed peaks identified as being oligomannose structures (peaks Man5, Man6, Man7, Man8 and Man9) by digestion with Jack Bean mannosidase (JBM). The relative amounts of these mannose structures changed between the first and eighth day of diseases and also in comparison with the control serum (FIG. 6). The amount of FA2B in the peak which co-elutes with Man5 was subtracted before comparisions between the mannose peaks were calculated for FIG. 6B.

Changes in the Degree of Branching

The degree of branching was measured after treating the glycan pool with a combination of a sialidase from Arthrobacter ureafaciens (ABS), β-galactosidase from S. pneumoniae (SPG) and α-fucosidase from bovine kidney (BKF), which reduced the glycans into the core antenna structures (GlcNAc only attached to the trimannosyl chitobiose core mono-, bi-, tri- and tetraantennary structures). As shown in FIG. 7A, the degree of branching in pancreatitis and sepsis was different from the control serum and also changed during the course of the diseases.

Other Glycans

The control and sepsis samples also contained low levels of glycans with an N-acetyl-lactosamine extension as detected by negative ion MS/MS. The spectra contained a very abundant F-type ion at m/z 789 confirming the antenna structure as Hex₂HexNAc₂. The high relative abundance of this ion is consistent with N-acetyl-lactosamine substitution on the 6-branch of the 6-antenna (reference Davids paper). Both sepsis Day 1 and Day 8 had an additional N-acetyl-lactosamine-extended glycan with an additional fucose. The spectrum from Day 1 was weak but that from Day 8 showed 80% of the fucosylated structures had outer-arm-fucose substitution in one of the antennae, and an F ion at m/z 935 (m/z 789+146) showed that at least some of this fucose was located on the N-acetyl-lactosamine-extended antenna. This compound was not detected in the early pancreatitis sample but was present on Day 8.

Discussion

The results demonstrate that changes of serum glycans occur very early in acute inflammation. The proportions of different glycans changed daily; some of them continuously in the same direction, while others varied during the course of acute pancreatitis and sepsis. The most prominent changes that consistently followed disease progression were observed for tri- and tetrasialylated structures as well as for oligomannose structures. These structures were also found to be altered in the first day of both pancreatitis and sepsis (when compared to the control serum).

In sepsis, the proportion of the trisialylated triantennary A3G3S3 in total glycan pool constantly decreased while in pancreatitis the proportion of the sialyl Lewis X structure A3FG3S3 constantly increased. The acute-phase protein α1-acid glycoprotein, which is elevated early in the acute-phase response, has been recognized as a principal carrier of this Lewis antigen (Brinkman-van der Linden, E. C., de Haan, P. F., et al. 1998). Haptoglobin and α1-antichymotripsyn were also found to contribute to the elevation of sialyl-Lewis X, but to lesser extent. Earlier studies also reported elevation of the expression of sialyl-Lewis X on α1-acid glycoprotein induced by inflammation, independent of the increase in the concentration of α1-acid glycoprotein (Higai, K., Aoki, Y., et al. 2005). A similar observation was made in malignant diseases (Croce, M. V., Salice, V. C., et al. 2005). It has been postulated that this increase might have beneficial effects by protecting the organism from overreaction that can occur during inflammation and which could be fatal (Bone, R. C. 1996). Since the enzyme responsible for the addition of fucose to A3G3S3 is α1-3 fucosyltransferase, the levels of this enzyme are crucial. A study on α1-acid glycoprotein suggested that inflammatory cytokines regulate the expression of α1-3 fucosyltransferase VI responsible for α1-3 fucosylation in liver tissue (De Graaf, T. W., Van der Stelt, M. E., et al. 1993, Higai, K., Aoki, Y., et al. 2005) as well as the expression of α2-3 sialyltransferase required for sialyl-Lewis X formation (since only structures containing α2-3-linked Neu5Ac can be fucosylated). Work on the prognostic value of α1-acid glycoprotein glycosylation in septic shock (Brinkman-van der Linden, E. C., van Ommen, E. C., et al. 1996), indicated that a modest elevation in biantennary glycans in combination with a strong increase in sialyl-Lewis X was associated with higher mortality than a high transient increase in biantennary with gradually increasing sialyl-Lewis X expression. This clearly demonstrates that the manner of changes in glycan structures can be associated with the severity of a disease.

The amounts of oligomannose structures were found to constantly decrease with the progression of acute pancreatitis, while in sepsis they varied slightly throughout the days. However, in both diseases these structures were markedly increased on Day 1 (compared to control) and then decreased on Day 8 (Man6 and Man9 fell to below the control level). These types of glycan structures can be found on the C3 component of complement (Hirani, S., Lambris, J. D., et al. 1986) which is also one of the positive acute-phase proteins. The complement pathway is derived from many small plasma proteins that form the biochemical cascade of the immune system. It is designed to destroy infectious microbes and damaged host material (Ritchie, G. E., Moffatt, B. E., et al. 2002). In contrast to most of the components synthesized mainly in the liver that have complex biantennary structures, C3 contains only oligomannose types (Hase, S., Kikuchi, N., et al. 1985, Hirani, S., Lambris, J. D., et al. 1986) with predominantly Man8 and Man9 on the a chain and Man6 on the β chain.

Increases in biantennary glycans have been reported in patients with acute and chronic inflammatory conditions as well as in cancer (Higai, K., Aoki, Y., et al. 2005). These compounds were also elevated, especially in the later stage of the acute response. In acute pancreatitis, biantennary glycans with bisecting GlcNAc were markedly elevated compared to the control. Tetraantennary structures were elevated in both diseases, although this elevation was more prominent in pancreatitis. Enzymes responsible for synthesising antennae on N-glycans are N-acetylglucosaminyl transferases (GnT). GnT III is the enzyme responsible for adding β-GlcNAc to the 4-position of the mannose in the core of N-glycans forming a bisecting β1→4-GlcNAc structure. GnT IV is responsible for forming triantennary structures by adding β1→4GlcNAc to the 3-antenna of the tri-mannosyl-chitobiose core while tetraantennary glycans are produced by subsequent actions by this enzyme and GnT V that adds β-GlcNAc to the 6-position of the mannose of the 6-antenna (Brockhausen, I., Hull, E., et al. 1989). Increased activities of these enzymes have been reported in many human malignancies (Dennis, J. W. and Laferte, S. 1989, Guo, J. M., Zhang, X. Y., et al. 2001, Jin, X. L., Liu, H. B., et al. 2004, Takamatsu, S., Oguri, S., et al. 1999, Yao, M., Zhou, D. P., et al. 1998). The results suggest an increase in the activity of these enzymes in the acute-phase response, especially in pancreatitis. An isoform of the triantennary glycans with a branched 6-antenna represents a significant proportion of the triantennary structures in both sepsis and pancreatitis, while in normal serum the amount of this structure is almost negligible (Table 2).

Comparison of tri- and tetra-antennary structures reveals that the ratio of α1-3-fucosylated forms to core fucosylated or non-fucosylated forms was increased in both pancreatitis and sepsis. This alteration in core fucosylation is different from the findings in different human cancers (Block, T. M., Comunale, M. A., et al. 2005, Ito, Y., Miyauchi, A., et al. 2003), as well as in pancreatic cancer (Barrabes, S., Pages-Pons, L., et al. 2007), where the increase in core fucosylation was observed and even suggested as a new diagnostic and prognostic marker. These changes in fucose levels can be a part of the regulatory processes during inflammation since it was suggested that it participates in immune modulation (Bone, R. C. 1996) (Listinsky, J. J., Listinsky, C. M., et al. 2001).

In general, the results show that changes of serum glycans can be observed very early in acute inflammation and that the proportions of different structures change daily. Since glycan profiles in healthy serum are more or less constant, these changes apparently mirror the disease. The observed differences between sepsis and acute pancreatitis are probably due to the fact that, in these two conditions, the acute-phase response is triggered by different stimuli and is, thus, associated with different patterns of production of specific cytokines. This complexity is not surprising knowing how complex and diverse the acute-phase response is and how important roles glycans play in many different processes.

All documents referred to in this specification are herein incorporated by reference. Various modifications and variations to the described embodiments of the inventions will be apparent to those skilled in the art without departing from the scope of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the art are intended to be covered by the present invention.

Abbreviations

2-AB—2-aminobenzamide; ABS—sialidase from Arthrobacter ureafaciens; AMF—Almond meal α-fucosidase; BKF—α-fucosidase from bovine kidney; BTG—β-galactosidase from bovine testes; CRP—C-reactive protein; DHB—dihydroxybenzoic acid; ESI—electrospray ionization; GnT—N-acetylglucosaminyl transferase; GU—glucose unit; GUH—β-N-acetyl-hexosaminidase; HPLC—high-performance liquid chromatography; JBM—Jack Bean α-mannosidase; MALDI—matrix-assisted laser desorption/ionization; MS—mass spectrometry; NAN1—neuraminidase from Streptococcus pneumoniae; NP—normal-phase; BTG—β-galactosidase from bovine testes; SPG—β-galactosidase from S. pneumoniae; BKF—α-fucosidase from bovine kidney; GUH—β-N-acetyl-hexosaminidase; JBM—Jack Bean α-mannosidase; AMF—Almond meal α-fucosidase; PNGase F—N-glycosidase F, QTOF—quadripole time-of-flight; SPG—β-galactosidase from S. pneumoniae; WAX—weak anion exchange; XMF—α-fucosidase from Xanthomonus sp.

Glycan structures are abbreviated as follows:

Ax, number of antenna (GlcNAc) on trimannosyl core; F at the start of the abbreviation indicates a core fucose α1-6 linked to the inner GlcNAc; Mx, number (x) of mannose on the core GlcNAc; Gx, number (x) of β31-4 linked galactose on antenna; G1[3] and G1[6] indicates that the galactose is on the antenna of the α1-3 or α1-6 mannose; F(x), number (x) of fucose linked α1-3 to antenna GlcNAc; Lac(x), number (x) of lactosamine (Galβ1-4GlcNAc) extensions; Sx, number (x) of sialic acids linked to galactose; the numbers 3 or 6 in parentheses after S indicate whether the sialic acid is in an α2-3, α2-6 linkage.

### REFERENCES

Barrabes, S., et al. (2007) Glycobiology, 17, 388-400.

Block, T. M., et al. (2005) Proc Natl Acad Sci USA, 102, 779-784.

Bond, A., et al. (1997) J Autoimmun, 10, 77-85.

Bone, R. C. (1996) Crit Care Med, 24, 163-172.

Börnsen, K. O., et al. (1995) Rapid Commun Mass Spectrom, 9, 1031-1034.

Brinkman-van der Linden, et al. (1998) Glycoconj J, 15, 177-182.

Brinkman-van der Linden, E. C., et al. (1996) Glycoconj J, 13, 27-31.

Brockhausen, I. et al. (1989) J Biol Chem, 264, 11211-11221.

Croce, M. V. et al. (2005) Histol Histopathol, 20, 91-97.

De Graaf, T. W., et al. (1993) J Exp Med, 177, 657-666.

Dennis, J. W. and Laferte, S. (1989) Cancer Res, 49, 945-950.

Gabay, C. and Kushner, I. (1999) N Engl J Med, 340, 448-454.

Guo, J. M. et al. (2001) J Cancer Res Clin Oncol, 127, 512-519.

Harvey, D. J. (1993) Rapid Commun Mass Spectrom, 7, 614-619.

Harvey, D. J. (2005) J. Am. Soc. Mass Spectrom, 16, 622-630 (Part 1), 631-646 (Part 2) and 647-659 (Part 3).

Hase, S. et al. (1985) J Biochem (Tokyo), 98, 863-874.

Higai, K. et al. (2005) Biochim Biophys Acta, 1725, 128-135.

Hirani, S. et al. (1986) Biochem J, 233, 613-616.

Ito, Y. et al. (2003) Cancer Lett, 200, 167-172.

Jin, X. L. et al. (2004) Glycoconj J, 20, 399-406.

Listinsky, J. J. et al. (2001) Adv Anat Pathol, 8, 330-337.

Lowe, J. B. (2003) Curr Opin Cell Biol, 15, 531-538.

Piagnerelli, M. et al. (2005) Shock, 24, 48-52.

Ritchie, G. E. et al. (2002) Chem Rev, 102, 305-320-319.

Royle, L. et al. (2006) Methods Mol Biol, 347, 125-143.

Takamatsu, S. et al. (1999) Cancer Res, 59, 3949-3953.

Van Dijk, W., et al. (1998) Adv Exp Med Biol, 435, 145-150.

van Dijk, W. et al. (1995) Glycoconj J, 12, 227-233.

Yao, M. et al. (1998) J Cancer Res Clin Oncol, 124, 27-30.

1. A method for diagnosing and/or prognosticating sepsis and/or pancreatitis, the method comprising:
providing a test sample from a subject in need thereof;
determining in said sample a level of one or more markers selected from the group consisting of outer arm to core fucosylation on N-linked glycans, trisialylated N-linked glycans, tetrasialylated N-linked glycans, biantennary glycans, mannose structures on N-linked glycans, degree of branching of N-linked glycans, ratio of trisialylated N-linked glycan A3G3S3 to fucosylated N-linked glycan A3FG3S3 (sialyl Lewis x), ratio of alpha 1,3 fucosylated forms of N-linked glycans to core fucosylated or non-fucosylated forms of N-linked glycans, trisialylated triantennary N-linked glycan A3G3S3, sialyl Lewis x N-linked glycan structure A3FG3S3, A3FG1 derived from digestion of sialyl Lewis x on N-linked glycans, oligomannose structures on N-linked glycans, isoforms of triantennary glycans with a branched 6-antenna and core fucosylation of N-linked glycans; and
providing a diagnosis and/or prognosis for said subject based on said determined level of the one or more markers;
wherein the diagnosis confirms the presence or absence of sepsis or pancreatitis in the subject and the prognosis confirms the possibility of the subject developing sepsis or pancreatitis.

2. A method as claimed in claim 1, wherein the sepsis is caused by a gram positive bacteria.

3-6. (canceled)

7. A method for monitoring a response to a sepsis or pancreatitis therapy comprising:
providing a first test sample from a subject in need thereof obtained at a first time point;
determining in said first sample a first level of one or more markers selected from the group consisting of outer arm to core fucosylation on N-linked glycans, trisialylated N-linked glycans, tetrasialylated N-linked glycans, biantennary glycans, mannose structures on N-linked glycans, degree of branching of N-linked glycans, ratio of trisialylated N-linked glycan A3G3S3 to fucosylated N-linked glycan A3FG3S3 (sialyl Lewis x), ratio of alpha 1,3 fucosylated forms of N-linked glycans to core fucosylated or non-fucosylated forms of N-linked glycans, trisialylated triantennary N-linked glycan A3G3S3, sialyl Lewis x N-linked glycan structure A3FG3S3, A3FG1 derived from digestion of sialyl Lewis x on N-linked glycans, oligomannose structures on N-linked glycans, isoforms of triantennary glycans with a branched 6-antenna and core fucosylation of N-linked glycans;
providing at least one further test sample from said subject at a second time point that is after said first time point, wherein the subject received a sepsis or pancreatitis therapy between the first time point and the second time point;
determining in the at least one further test sample a second level of the one or more markers;
assessing a response of the subject to the sepsis or pancreatitis therapy based on a comparison of the first determined level of the one or more markers and the second determined level of the one or more markers.

8-12. (canceled)

13. A method for prognosticating or diagnosing pancreatic cancer, the method comprising:
providing a test sample from a subject in need thereof;
determining in said sample a level of one or more markers selected from the group consisting of outer arm to core fucosylation on N-linked glycans, trisialylated N-linked glycans, tetrasialylated N-linked glycans, biantennary glycans, mannose structures on N-linked glycans, degree of branching of N-linked glycans, ratio of trisialylated N-linked glycan A3G3S3 to fucosylated N-linked glycan A3FG3S3 (sialyl Lewis x), ratio of alpha 1,3 fucosylated forms of N-linked glycans to core fucosylated or non-fucosylated forms of N-linked glycans, trisialylated triantennary N-linked glycan A3G3S3, sialyl Lewis x N-linked glycan structure A3FG3S3, A3FG1 derived from digestion of sialyl Lewis x on N-linked glycans, oligomannose structures on N-linked glycans, isoforms of triantennary glycans with a branched 6-antenna or core fucosylation of N-linked glycans; and
providing a prognosis and/or diagnosis for the subject based on said determined level of the one or more markers;
wherein the diagnosis confirms the presence or absence of pancreatic cancer in the subject and the prognosis confirms a possibility of the subject developing pancreatic cancer.

14. (canceled)

15. A method as claimed in claim 1, wherein said providing the diagnosis and/or prognosis comprises providing for the subject a sepsis diagnosis, that confirms presence or absence of sepsis in said subject.

16. A method as claimed in claim 1, wherein said providing the diagnosis and/or prognosis comprises providing for the subject a sepsis prognosis, that confirms a possibility for developing sepsis in said subject.

17. A method as claimed in claim 1, wherein said providing the diagnosis and/or prognosis comprises providing for the subject a pancreatitis diagnosis, that confirms presence or absence of pancreatitis in said subject.

18. A method as claimed in claim 1, wherein said providing the diagnosis and/or prognosis comprises providing for the subject a pancreatitis prognosis, that confirms a possibility for developing pancreatitis in said subject.

19. A method as claimed in claim 1, wherein the sample comprises whole serum of the subject.

20. A method as claimed in claim 1, wherein said providing the diagnosis and/or prognosis comprises comparing the level of the one or more markers in the test sample with a level of the one or more markers in a control sample.

21. A method as claimed in claim 20, wherein said providing the diagnosis and/or prognosis comprises determining a presence of pancreatitis or sepsis in the subject based on one of the following:
a) an increase, compared to the level in the control sample, in the determined level in the test sample of at least one of the ratio of outer arm to core fucosylation on N-linked glycans, tetrasialylated N-linked glycans, biantennary glycans, the ratio of alpha 1,3 fucosylated forms of N-linked glycans to core fucosylated or non-fucosylated forms of N-linked glycans, isoforms of triantennary glycans with a branched 6-antenna, sialyl Lewis x N-linked structure glycan A3FG3S3 and A3FG1 derived from digestion of sialyl Lewis x on N-linked glycans;
b) changes, compared to the level in the control sample, in the determined level in the test sample of mannose structures on N-linked glycans and
c) changes, compared to the level in the control sample. in the determined level in the test sample of the degree of branching of N-linked glycans.

22. A method as claimed in claim 20, wherein said providing the diagnosis and/or prognosis comprises determining a presence of pancreatitis or sepsis in the subject based on a decrease, compared to the level of the control sample, in the determined level in the test sample in at least one of the trisialylated triantennary N-linked glycan A3G3S3 and the ratio of trisialylated N-linked glycan A3G3S3 to fucosylated N-linked glycan A3FG3S3 (sialyl Lewis x) indicates the presence of pancreatitis or sepsis.

23. A method as claimed in claim 20, wherein said providing the diagnosis and/or prognosis comprises determining a presence of pancreatitis in the subject based on a continuous increase in the level of the sialyl Lewis x N-linked glycan structure A3FG3S3 and/or a continuous decrease in the level of oligomannose structures indicates the presence of pancreatitis.

24. A method as claimed in claim 1, wherein said determining comprises determining levels of two or more of the one or more markers and said providing the diagnosis and/or prognosis comprises providing the diagnosis and/or prognosis based on said determined levels of the two or more markers.

25. A method as claimed in claim 1, further comprising
determining in said test sample or in an additional sample from the subject a level of one or more additional markers comprising at least one of a genetic marker and a protein marker;
and providing the diagnosis and/or prognosis based on said determined level of the at least one marker and the determined level of the one or more additional markers.

26. A method as claimed in claim 7, wherein the subject received a sepsis therapy and said assessing comprises assessing a response of the subject to said sepsis therapy.

27. A method as claimed in claim 7, wherein the subject received a pancreatitis therapy and said assessing comprises assessing a response of the subject to said pancreatitis therapy.

28. A method as claimed in claim 7, wherein each of the first sample and the at least one further test sample comprises whole serum of the subject.

29. A method as claimed in claim 7, wherein said determining in said first sample comprises determining first levels of two or more markers of the one or more markers, said determining in the at least one further sample comprises determining second levels of the two or more markers and said assessing comprises assessing the response of the subject to the sepsis or pancreatitis therapy based on a comparison of said determined first levels and said determined second levels of the two or more markers.

30. A method as claimed in claim 7, further comprising
determining in said sample or in an additional sample from the subject a level of one or more additional markers comprising at least one of a genetic marker and a protein marker;
and assessing the response of the subject to the sepsis or pancreatitis therapy based on said determined levels of the at least one marker and the one or more additional markers.
